(19) United States
(12) Patent Application Publication
(10) Pub. No.: US 2025/0232866 A1
(43) Pub. Date: Jul. 17, 2025

(54) RADIATION IMAGING SYSTEM, CONTROL METHOD THEREFOR, AND STORAGE MEDIUM

(71) Applicant: CANON KABUSHIKI KAISHA, Tokyo (JP)

(72) Inventor: SHINSUKE HAYASHIDA, Tokyo (JP)

(21) Appl. No.: 19/013,612

(22) Filed: Jan. 8, 2025

(30) Foreign Application Priority Data
Jan. 11, 2024 (JP) 2024-002797

(51) Int. Cl. G16H 30/40; A61B 6/46; A61B 6/58

(52) U.S. Cl. CPC G16H 30/40; A61B 6/465; A61B 6/585

(57) ABSTRACT

A radiation imaging system includes one or more controllers that acquire information about a radiation irradiation range for radiographic imaging and cause at least one correction image from a plurality of correction images different in irradiation condition of radiation to be displayed on a display in a selectable manner based on the information about the radiation irradiation range for radiographic imaging.

### BACKGROUND

### Field

Aspects of the present disclosure generally relate to a radiation imaging system, a control method therefor, and a storage medium.

### Description of the Related Art

Some practically used radiation imaging apparatuses (radiation imaging units) for use in medical imaging diagnosis or non-destructive inspection using radiation such as X-rays include a configuration in which pixels each of which includes a switch element, such as a thin-film transistor (TFT), and a conversion element, such as a photoelectric conversion element, using semiconductors, are arranged in a matrix pattern. In the radiation imaging apparatuses (radiation imaging units) using semiconductors, image processing such as gain correction processing or gradation processing is performed on a radiographic image obtained by image capturing and image display is able to be performed several seconds after irradiation of radiation for image capturing. In a product lineup of radiation imaging apparatuses (radiation imaging units), sizes standardized by, for example, conventional films or computed radiography (CR) have been used. Examples of the largest sizes from standard sizes of radiation imaging apparatuses (radiation imaging units) include “Ohban (Large Size)” (about 17 inches×about 17 inches) and “Hansetsu (Half-cut)” (about 14 inches×about 17 inches). Then, in a case where a more large-sized radiation imaging apparatus (radiation imaging unit) is required, there can be used a method of utilizing a conventional standardized size and, for example, performing image capturing with a plurality of radiation imaging apparatuses used in an overlapping manner and then performing image correction processing on overlapped areas, as discussed in Japanese Patent Application Laid-Open No. 2016-198424. In this way, recently, there has been a need for an increase in size of a radiation imaging apparatus (radiation imaging unit).

On the other hand, on a radiation generation device (radiation generation unit) which generates radiation used for radiographic imaging, a radiation movable diagram (radiation diagram) is mounted in such a manner that only a minimum necessary range is irradiated with radiation and the other ranges are shielded from radiation. With the radiation movable diagram used, there are restrictions on the maximum irradiation field (for example, “JIS Z 4712:1998 Guide”), so that restrictions are imposed so as to prevent radiation from being radiated to sizes larger than or equal to the above-mentioned standardized size due to the arrangement of the radiation generation device at the time of image capturing.

If, in this way, although the irradiation range of radiation radiated by the radiation generation device is restricted by the radiation movable diaphragm, the radiation imaging apparatus is made larger in size, there may be a case where it is impossible to radiate radiation to the whole area of the radiation imaging apparatus (radiation imaging unit) at a field site such as medical facility. In this case, there is an issue in which it may be difficult to appropriately correct a radiographic image captured by the radiation imaging apparatus (radiation imaging unit) based on incident radiation.

### SUMMARY

Aspects of the present disclosures are generally directed to providing a technique that corrects a captured radiographic image even in a case where it is impossible to radiate radiation to the whole area of a radiation imaging unit.

According to an aspect of the present disclosure, a radiation imaging system includes one or more controllers configured to acquire information about a radiation irradiation range for radiographic imaging, and cause at least one correction image from a plurality of correction images different in irradiation condition of radiation to be displayed on a display in a selectable manner based on the information about the radiation irradiation range for radiographic imaging.

Further features of the present disclosure will become apparent from the following description of exemplary embodiments with reference to the attached drawings.

### BRIEF DESCRIPTION OF THE DRAWINGS

FIG. 1 is a diagram illustrating an example of an outline configuration of a radiation imaging system according to a first exemplary embodiment.

FIG. 2A is a flowchart illustrating an example of a processing procedure performed at the time of factory shipment in a control method for the radiation imaging system according to the first exemplary embodiment.

FIG. 2B is a flowchart illustrating an example of a processing procedure performed at the time of installation or at the time of periodic inspection in the control method for the radiation imaging system according to the first exemplary embodiment.

FIG. 2C is a flowchart illustrating an example of a processing procedure performed at the time of use in installation location in the control method for the radiation imaging system according to the first exemplary embodiment.

FIGS. 3A and 3B are diagrams used to explain a radiation irradiation range (radiation irradiation field), which is an irradiation range of radiation, in a radiation imaging unit according to the first exemplary embodiment.

FIGS. 4A and 4B are diagrams illustrating an example of installation of the radiation imaging unit according to the first exemplary embodiment.

FIGS. 5A and 5B are diagrams illustrating examples of screens each of which can be displayed on a display unit in the radiation imaging system according to the first exemplary embodiment.

FIGS. 6A, 6B, and 6C are diagrams used to explain an example of an application in which it is supposed to increase the image quality by displaying not only information about a radiation irradiation range but also ancillary information in the radiation imaging system according to the first exemplary embodiment.

FIG. 7A is a diagram illustrating examples of screens which can be displayed on a display unit in a radiation imaging system according to a second exemplary embodiment.

FIG. 7B is a diagram illustrating examples of screens which can be displayed on the display unit in the radiation imaging system according to the second exemplary embodiment.

FIG. 8 is a flowchart illustrating an example of a processing procedure performed at the time of use in installation location in a control method for the radiation imaging system according to the second exemplary embodiment.

FIG. 9A is a diagram illustrating examples of screens that can be displayed on a display unit in a radiation imaging system according to a third exemplary embodiment.

FIG. 9B is a diagram illustrating examples of screens which can be displayed on the display unit in the radiation imaging system according to the third exemplary embodiment.

FIG. 10 is a flowchart illustrating an example of a processing procedure performed at the time of use in installation location in a control method for the radiation imaging system according to the third exemplary embodiment.

FIG. 11A is a diagram illustrating a first configuration example of a control system for a radiation imaging system according to a fourth exemplary embodiment.

FIG. 11B is a diagram illustrating a second configuration example of the control system for the radiation imaging system according to the fourth exemplary embodiment.

FIG. 12 is a flowchart illustrating an example of a processing procedure in a control method for the radiation imaging system according to the fourth exemplary embodiment.

FIG. 13 is a flowchart illustrating an example of a processing procedure performed at the time of use in installation location in the control method for the radiation imaging system according to the fourth exemplary embodiment.

FIG. 14 is a diagram illustrating examples of application of a radiation imaging system according to a fifth exemplary embodiment.

### DESCRIPTION OF THE EMBODIMENTS

Various exemplary embodiments, features, and aspects of the disclosure will be described in detail below with reference to the drawings. Furthermore, while it is favorable to use X-rays as radiation described in the present disclosure, radiation is not limited to X-rays, but can also include, for example, alpha (α) rays, beta (β) rays, and gamma (β) rays.

First, a first exemplary embodiment is described.

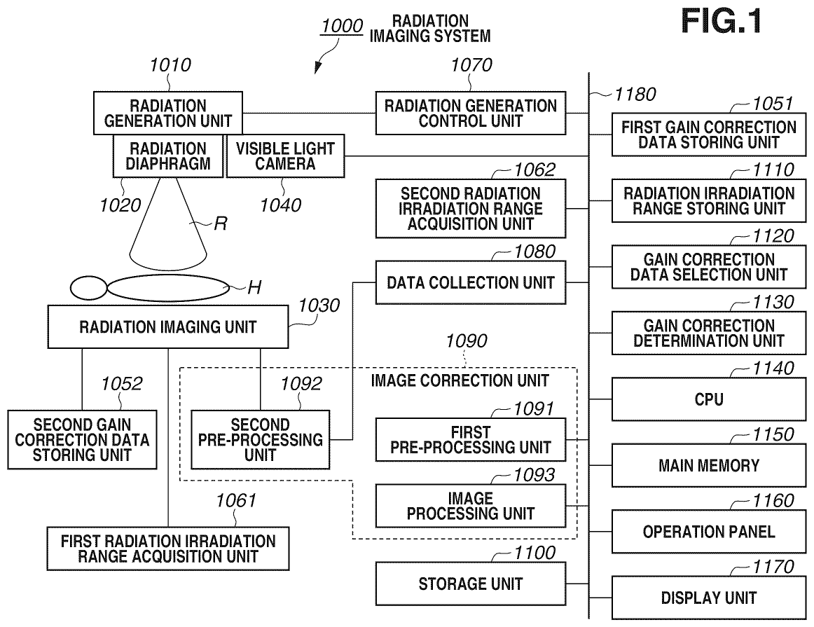

FIG. 1 is a diagram illustrating an example of an outline configuration of a radiation imaging system 1000 according to the first exemplary embodiment. The radiation imaging system 1000 in the first exemplary embodiment is used mainly for medical purposes.

The radiation imaging system 1000 in the first exemplary embodiment includes a radiation generation unit 1010, a radiation diaphragm 1020, a radiation imaging unit 1030, a visible light camera 1040, a first gain correction data storing unit 1051, and a second gain correction data storing unit 1052. Additionally, the radiation imaging system 1000 in the first exemplary embodiment includes a first radiation irradiation range acquisition unit 1061, a second radiation irradiation range acquisition unit 1062, a radiation generation control unit 1070, a data collection unit 1080, an image correction unit 1090, and a storage unit 1100. Additionally, the radiation imaging system 1000 in the first exemplary embodiment includes a radiation irradiation range storing unit 1110, a gain correction data selection unit 1120, a gain correction determination unit 1130, a central processing unit (CPU) 1140, a main memory 1150, an operation panel 1160, a display unit 1170, and a bus 1180.

The radiation generation unit 1010 radiates radiation R toward a subject H and the radiation imaging unit 1030 via the radiation diaphragm 1020 under the control of the radiation generation control unit 1070.

The radiation diaphragm 1020 restricts the irradiation range of radiation R radiated from the radiation generation unit 1010 (radiation irradiation range or radiation irradiation field). The radiation diaphragm 1020 is internally provided with a light source which radiates visible light, and turning on the light source enables a medical worker to recognize an approximate radiation irradiation range based on an irradiation range of visible light having passed through the radiation diaphragm 1020 (light irradiation range or light irradiation field). The radiation imaging unit 1030 captures a radiographic image based on incident radiation R (including radiation R having penetrated the subject H). The radiation imaging unit 1030 is a large-sized, for example, elongated, imaging unit, and, in the first exemplary embodiment, a case where radiation R is not radiated to the whole area of an entrance surface on which radiation R is incident is also supposed. The visible light camera 1040 is mounted to, for example, the radiation diaphragm 1020, and is set in such a way as to be able to perform image capturing of the whole region of the radiation imaging unit 1030.

Each of the first gain correction data storing unit 1051 and the second gain correction data storing unit 1052 stores gain correction data (data for gain correction) including a gain correction image (an image for gain correction) for correcting a difference in gain of the radiation imaging unit 1030. For example, the gain correction image is acquired by radiating radiation R for gain correction to the radiation imaging unit 1030 in a state in which the subject H is not present. Furthermore, while, in the example illustrated in FIG. 1, two gain correction data storing units 1051 and 1052 are provided, in the first exemplary embodiment, a configuration in which only one gain correction data storing unit is provided can be employed. In the first exemplary embodiment, the gain correction data storing unit stores a plurality of pieces of gain correction data including a plurality of correction images (images for correction) different in irradiation condition of radiation R.

Each of the first radiation irradiation range acquisition unit 1061 and the second radiation irradiation range acquisition unit 1062 acquires a radiation irradiation range for image capturing, which is an irradiation range of radiation R when a radiographic image has been captured by the radiation imaging unit 1030. Furthermore, while, in the example illustrated in FIG. 1, two radiation irradiation range acquisition units 1061 and 1062 are provided, in the first exemplary embodiment, a configuration in which only one radiation irradiation range acquisition unit is provided can be employed.

The radiation generation control unit 1070 controls irradiation of radiation R generated by the radiation generation unit 1010 under the control of, for example, the CPU 1140. The data collection unit 1080 collects gain correction data from the first gain correction data storing unit 1051, collects data about a radiation irradiation range for image capturing from the first radiation irradiation range acquisition unit 1061, and collects corrected image data from the image correction unit 1090. The image correction unit 1090 corrects a radiographic image using a correction image (an image for correction) selected from at least one correction image displayed by the display unit 1170 in a selectable manner. The image correction unit 1090 is configured to include, as illustrated in FIG. 1, a first pre-processing unit 1091, a second pre-processing unit 1092, and an image processing unit 1093. The storage unit 1100 stores, for example, various pieces of information (including data) obtained as a result of the CPU 1140 performing various control operations or various processing operations.

The radiation irradiation range storing unit 1110 stores radiation irradiation ranges for correction, which are irradiation ranges of radiation R when respective gain correction images for a plurality of gain correction images have been acquired in the radiation imaging unit 1030. The gain correction data selection unit 1120 selects gain correction data to be used from among a plurality of pieces of gain correction data including a plurality of gain correction images. The gain correction determination unit 1130 performs various determinations concerning gain correction data including a gain correction image.

The CPU 1140 performs control of the radiation imaging system 1000, and performs various processing operations. The main memory 1150 stores, for example, various pieces of information (including data) and programs which are required when the CPU 1140 performs various control operations and various processing operations. Furthermore, a configuration in which various pieces of information (including data) and programs described here as being stored in the main memory 1150 are stored in, for example, the storage unit 1100 can be employed. The operation panel 1160 is used for a user such as a medical worker to perform operation input. Here, information obtained by performing operation input via the operation panel 1160 is input to, for example, the CPU 1140 and is then processed thereby. The display unit 1170 displays various pieces of information (including data) and various images under the control of, for example, the CPU 1140. The bus 1180 interconnects respective connected constituent units in such a manner that the constituent units communicate with each other.

Upon receipt of an image capturing order, the user such as a medical worker operates the operation panel 1160 to input and set an image capturing condition. Here, the image capturing order includes inputs of, for example, an image capturing region, build, and age of the subject H and an image capturing purpose. The set image capturing condition is set to the radiation generation unit 1010 and the radiation imaging unit 1030, which includes a radiation detection unit equipped with a plurality of pixels in a two-dimensional surface manner, via the CPU 1140 and the bus 1180. Here, the image capturing condition to be set includes, for example, an X-ray tube voltage and tube current of the radiation generation unit 1010, an irradiation time of radiation R, the type of an anti-scatter grid, and the body position of the subject H. The user such as a medical worker performs positioning of the subject H and the radiation imaging unit 1030. The radiation imaging unit 1030 can be installed on an upright stand or can be installed in a recumbent table as illustrated in FIG. 1. In the case of a large-sized radiation imaging unit 1030, it is not essential to move the radiation imaging unit 1030 and the radiation generation unit 1010 only needs to be moved in such a way as to be able to irradiate the image capturing region of the subject H. The radiation R is narrowed down by the radiation diaphragm 1020 in such a manner that the radiation R is radiated only to a required range. The visible light camera 1040 is mounted to the radiation diaphragm 1020, thus enabling a range in which visible light radiated from the light source of the radiation diaphragm 1020 is radiated to the radiation imaging unit 1030 (light irradiation range) to be recognized via a camera image displayed on the display unit 1170.

The display unit 1170 displays a plurality of correction images including a recently acquired correction image together with a result of acquisition of an irradiation range (irradiation field) obtained by performing image analysis using these correction images as inputs. The user such as a medical worker compares a light irradiation range in a camera image captured by the visible light camera 1040 with radiation irradiation ranges for correction in a plurality of correction images via the display unit 1170 on which the camera image and the plurality of correction images are simultaneously displayed. Then, the user such as a medical worker checks whether the light irradiation range in the camera image is contained in a radiation irradiation range for correction acquired in a correction image selected from the plurality of correction images. If the light irradiation range is not contained in the radiation irradiation range for correction, the radiation imaging system 1000 performs navigation display, on the display unit 1170, of information indicating that effect and information about a deviation amount relative to the radiation irradiation range for correction in an excess range, which is a portion of the light irradiation range not contained in the radiation irradiation range for correction. Then, based on the information displayed on the display unit 1170, the user such as a medical worker moves the radiation generation unit 1010 or selects another correction image, and ends a preparation to be performed before irradiation of radiation R.

In the case of using a large-sized radiation imaging unit 1030, a gain correction image for correcting a difference in sensitivity of pixels is set selectable via the operation panel 1160. The position of a recently acquired gain correction image is displayed on the display unit 1170, and the gain correction image is selected in the operation panel 1160. Alternatively, the gain correction image is selected in the operation panel 1160 based on the light irradiation range in the camera image displayed on the display unit 1170.

The subject H is, for example, a human body. The radiation imaging unit 1030 is configured to include a radiation detection unit in which pixels each of which includes a phosphor for converting incident radiation R into light and a photoelectric conversion element for converting the light generated by the phosphor into an electrical signal (image signal) are arranged in a two-dimensional surface manner.

Here, the phosphors are formed in an effective pixel range (effective image capturing area) of the radiation imaging unit 1030. In the radiation imaging unit 1030, an electrical signal (image signal) obtained by the photoelectric conversion element is subjected to readout driving in a driving circuit and a readout circuit, is amplified after the readout driving, and is converted from an analog signal into a digital signal, thus becoming data about an image. The data about an image obtained by the radiation imaging unit 1030 is sent to the data collection unit 1080. The data about an image collected by the data collection unit 1080 is subjected to, for example, dark current correction processing, gain correction processing, and loss correction processing by the first pre-processing unit 1091 and the second pre-processing unit 1092, and is then subjected to, for example, quality assurance (QA) processing by the image processing unit 1093. Here, in the QA processing, for example, gradation processing, noise suppression processing, and frequency processing are performed. The image subjected to processing by the image correction unit 1090 finally becomes a diagnostic image, which is then displayed on the display unit 1170.

Furthermore, in the radiation imaging system 1000, for example, the CPU 1140 does not need to be mounted as a personal computer (PC), but can be mounted as a field-programmable gate array (FPGA) inside the radiation imaging unit 1030.

Next, a processing procedure in a control method for the radiation imaging system 1000 according to the first exemplary embodiment is described.

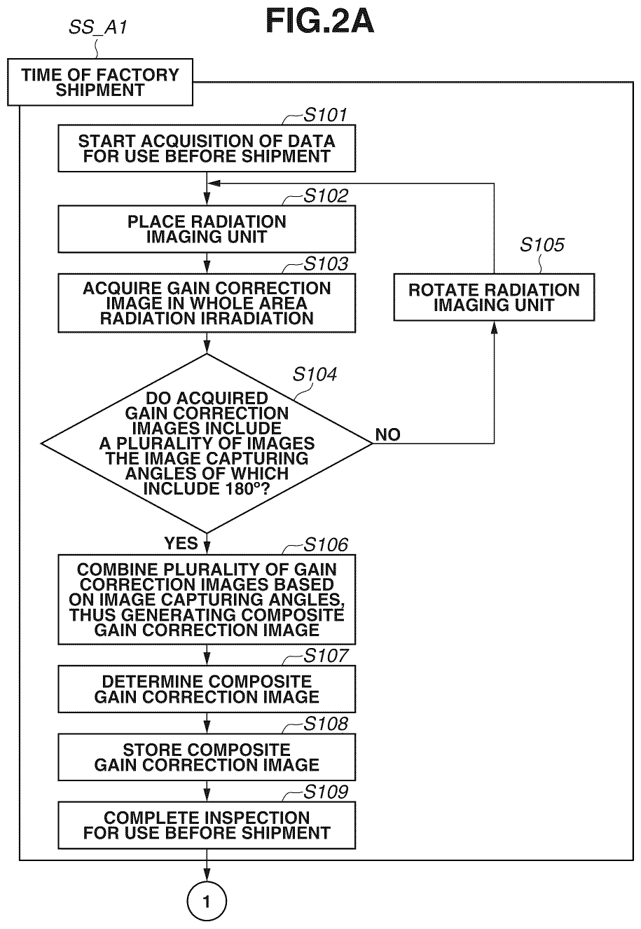

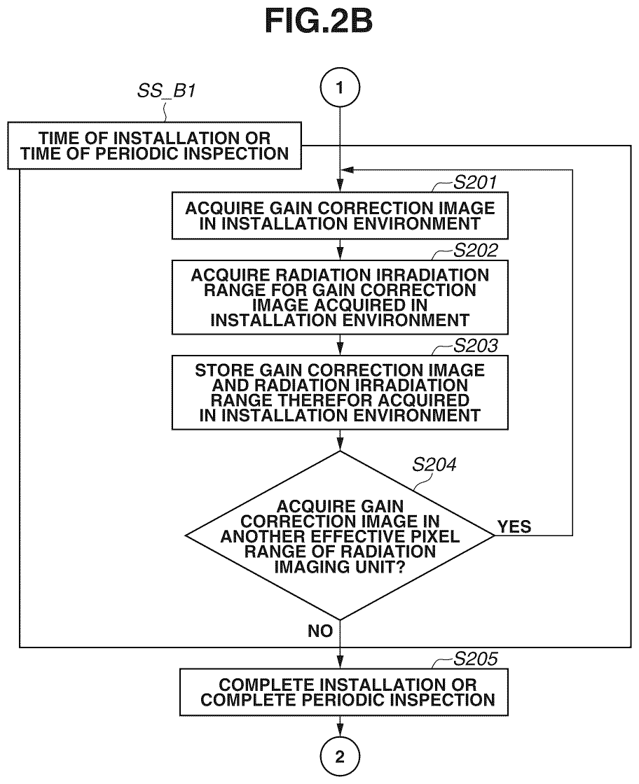

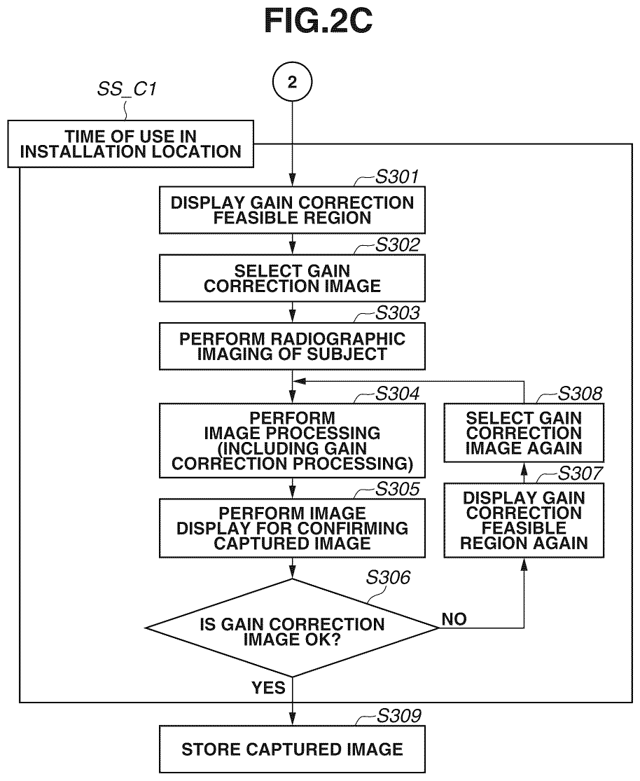

FIG. 2A is a flowchart illustrating an example of a processing procedure performed at the time of factory shipment in a control method for the radiation imaging system 1000 according to the first exemplary embodiment. In FIG. 2A, a flowchart illustrating an example of a processing procedure performed at the time of factory shipment in the first exemplary embodiment is denoted by SS_A1. Specifically, the flowchart (SS_A1) at the time of factory shipment illustrated in FIG. 2A is a flowchart for performing setting and inspection of setting before shipment of the radiation imaging unit 1030.

After the flowchart (SS_A1) at the time of factory shipment illustrated in FIG. 2A is started, first, in step S101, the radiation imaging unit 1030 starts acquisition of a correction image for use before shipment. Furthermore, in a case where, in, for example, medical facility, it is possible to radiate radiation R to the whole area of the radiation imaging unit 1030, the processing in the flowchart illustrated in FIG. 2A is not essential processing.

Next, in step S102, an inspector for the time of factory shipment places the radiation imaging unit 1030 at a predetermined position to acquire a correction image for use before shipment (in the first exemplary embodiment, a gain correction image). In a hospital or clinic being an installation location, there is a case where it is not possible to radiate radiation R to the whole area of the radiation imaging unit 1030 at one time. On the other hand, in a factory inspection for use before shipment, since it is possible to increase the distance between the radiation generation unit 1010 and the radiation imaging unit 1030, it is possible to radiate radiation R to the whole area of the radiation imaging unit 1030. To fulfill shipment in the universal state, it is desirable that the radiation generation unit 1010, which is used to acquire a gain correction image for use before shipment (in the first exemplary embodiment, a gain correction image), be a radiation generation unit that radiates radiation R as uniformly as possible and that has isotropic nature. In the case of using a widely used radiation generation unit 1010, uniformity and isotropic nature are improved by increasing the distance between the radiation generation unit 1010 and the radiation imaging unit 1030 and then acquiring correction images in a plurality of rotation angles in such a way as to cancel out un-uniformity of irradiation of radiation R caused by directions of an anode and a cathode.

Next, in step S103, the radiation imaging system 1000 causes the radiation generation unit 1010 to radiate radiation R to the whole area of the radiation imaging unit 1030 and first acquires a gain correction image at one image capturing position of the radiation imaging unit 1030. The radiation imaging system 1000 acquires a plurality of gain correction images as described below.

Next, in step S104, the radiation imaging system 1000 determines whether the gain correction images acquired in step S103 include a plurality of gain correction images the image capturing angles in the image capturing positions of which include 180°. Here, the image capturing angles refer to angles in upward, downward, leftward, and rightward directions of the radiation imaging unit 1030 as viewed from the anode-cathode direction of the radiation generation unit 1010.

The inspector for the time of factory shipment recognizes whether gain correction images have been acquired with relative angles of 0° and 180° on the same plane centering on the center of the radiation imaging unit 1030. Since there is a heel effect in the anode-cathode direction of the radiation generation unit 1010, this is performed to reduce the heel effect. In a case where the shape of an entrance surface on which radiation R is incident in the radiation imaging unit 1030 is not such a shape as a regular polygon, it is desirable to set the anode-cathode direction to the short side in the radiation generation unit 1010 and acquire gain correction images with relative angles of 0° and 180° on the same plane. The reason why it is desirable to acquire a plurality of images with the respective angles is because, since there is quantum noise in radiation R, if quantum noise is included in a correction image, quantum noise may appear in a frosted glass manner after correction of a diagnostic image.

If, in step S104, it is determined that the gain correction images acquired in step S103 do not include a plurality of gain correction images the image capturing angles in the image capturing positions of which includes 180° (NO in step S104), the radiation imaging system 1000 advances the processing to step S105. In step S105, the inspector for the time of factory shipment rotates the radiation imaging unit 1030. After that, the radiation imaging system 1000 returns the processing to step S102, thus performing processing operations in step S102 and subsequent steps again.

On the other hand, if in step S104, it is determined that the gain correction images acquired in step S103 include a plurality of gain correction images the image capturing angles in the image capturing positions of which includes 180° (YES in step S104), the radiation imaging system 1000 advances the processing to step S106. In step S106, the radiation imaging system 1000 combines the plurality of gain correction images based on the image capturing angles, thus generating a composite gain correction image. Here, it is desirable to, as a first stage, average gain correction images for each rotated angle and, after that, average gain correction images for the respective rotated angles and combine the averaged gain correction images. This is because, if all of the gain correction images are simply averaged and combined, as viewed from the anode-cathode direction of the radiation generation unit 1010, there may be a distribution of generation of radiation radiated from the radiation generation unit 1010 due to a difference in number of images for the respective rotation angles of the radiation imaging unit 1030.

Next, in step S107, the inspector for the time of factory shipment determines the gain correction image (the composite gain correction image). In the case of acquiring a gain correction image in a hospital or clinic being an installation location, since, even if, for example, dust has adhered to the surface of the radiation generation unit 1010, it is possibly to immediately remove the dust when using the radiation imaging system 1000 in the applicable environment, there may be no problem. However, with regard to a gain correction image for the time of factory shipment, in a case where, for example, flaw or dust is shown in an image, when the radiation imaging system 1000 is used in a hospital or clinic being an installation location, such flaw or dust may become shown in each image. Therefore, it is necessary to carry out an inspection before factory shipment. For the inspection method, it is desirable to use a qualitative evaluation, such as a visual evaluation, and a quantitative evaluation in a mutually complementary manner.

If the result of determination (inspection) in step S107 is OK, then in step S108, the radiation imaging system 1000 stores the composite gain correction image generated in step S106 in, for example, the second gain correction data storing unit 1052.

Next, in step S109, the inspector for the time of factory shipment completes an inspection for use before shipment.

Furthermore, in the case of a conventional radiation imaging unit having a standardized film size, the processing in a flowchart for the time of factory shipment can be omitted. However, in the case of a radiation imaging unit having a rectangular effective pixel range the length of the long side of which is 18 inches or more or the length of the diagonal of which is 25 inches or more, such as the radiation imaging unit 1030 in the first exemplary embodiment, if the processing in a flowchart for the time of factory shipment illustrated in FIG. 2A is not performed, an issue may occur. In the case of a radiation imaging unit standardized with 17 inches or less, in an installation location such as a hospital or clinic, it is possible to radiate radiation R to the whole area of the radiation imaging unit. Therefore, in the radiation imaging unit standardized with 17 inches or less, it is possible to reduce cost for a process before shipment by acquiring, after installation, a correction image compatible with an environment occurring after installation. On the other hand, in the case of the radiation imaging unit 1030 the length of the long side of which is 18 inches or more, since there are restrictions of, for example, the radiation diaphragm 1020 according to, for example, “JIS Z 4712:1998 Guide”, the processing in a flowchart for the time of factory shipment illustrated in FIG. 2A becomes necessary.

FIG. 2B is a flowchart illustrating an example of a processing procedure performed at the time of installation or at the time of periodic inspection in the control method for the radiation imaging system 1000 according to the first exemplary embodiment. In FIG. 2B, a flowchart illustrating an example of a processing procedure performed at the time of installation or at the time of periodic inspection in the first exemplary embodiment is denoted by SS_B1. The processing in the flowchart (SS_B1) at the time of installation or at the time of periodic inspection illustrated in FIG. 2B can be performed after the processing in the flowchart (SS_A1) at the time of factory shipment illustrated in FIG. 2A ends.

After the flowchart (SS_B1) at the time of installation or at the time of periodic inspection illustrated in FIG. 2B is started, first, in step S201, the radiation imaging system 1000 causes the radiation generation unit 1010 to radiate radiation R to the radiation imaging unit 1030 in an installation environment. Then, the radiation imaging system 1000 acquires a gain correction image based on radiation R radiated to the radiation imaging unit 1030. The radiation imaging unit 1030 having an effective pixel range the length of the long side of which is 18 inches or more is difficult to handle in terms of weight or size. Therefore, not demounting the radiation imaging unit 1030 from an upright stand or recumbent table becomes predominantly more frequent than in the case of a conventional radiation imaging unit with the standardized size of 17 inches or less. In a case where the radiation imaging unit 1030 is installed, the radiation imaging unit 1030 is placed on, for example, a recumbent table. Therefore, in the installation environment, acquiring a gain correction image enables correcting scattering radiation or unevenness specific to the installation environment. However, in a case where, for example, the radiation imaging unit 1030 having a rectangular effective pixel range the length of the long side of which is 18 inches or more or the length of the diagonal of which is 25 inches or more has been placed on a recumbent table, the following problem may occur. In other words, due to restrictions in the radiation diaphragm 1020, it may be impossible to acquire an appropriate gain correction image in the whole area of the effective pixel range.

Next, in step S202, the radiation imaging system 1000 acquires a radiation irradiation range for correction, which is an irradiation range of radiation R used for acquiring a gain correction image acquired in step S201 in the radiation imaging unit 1030. On this occasion, it is favorable to acquire the radiation irradiation range for correction with use of an irradiation field recognition function in image processing performed on the gain correction image, or the radiation irradiation range for correction can be acquired with a method in which the user performs 4-point instructions with respect to the displayed gain correction image.

Next, in step S203, the radiation imaging system 1000 stores the gain correction image in, for example, the second gain correction data storing unit 1052 and stores the radiation irradiation range for correction in the radiation irradiation range storing unit 1110 while associating the gain correction image and the radiation irradiation range for correction with each other. Furthermore, since the second gain correction data storing unit 1052 also has a memory capacity, a configuration in which the gain correction image is also stored in the first gain correction data storing unit 1051 and can be called by, for example, an operation input to the operation panel 1160 can be employed.

Next, in step S204, the radiation imaging system 1000 determines whether to acquire a gain correction image in another effective pixel range of the radiation imaging unit 1030 based on, for example, an operation input to the operation panel 1160. If, in step S204, it is determined to acquire a gain correction image in another effective pixel range of the radiation imaging unit 1030 (YES in step S204), the radiation imaging system 1000 returns the processing to step S201, thus performing processing operations in step S201 and subsequent steps in another installation environment.

On the other hand, if, in step S204, it is determined not to acquire a gain correction image in another effective pixel range of the radiation imaging unit 1030 (NO in step S204), the radiation imaging system 1000 advances the processing to step S205. In step S205, the user completes installation or completes periodic inspection.

FIG. 2C is a flowchart illustrating an example of a processing procedure performed at the time of use in installation location in the control method for the radiation imaging system 1000 according to the first exemplary embodiment. In FIG. 2C, a flowchart illustrating an example of a processing procedure performed at the time of use in installation location in the first exemplary embodiment is denoted by SS_C1. The processing in the flowchart (SS_C1) at the time of use in installation location illustrated in FIG. 2C can be performed after the processing in the flowchart (SS_B1) at the time of installation or at the time of periodic inspection illustrated in FIG. 2B ends.

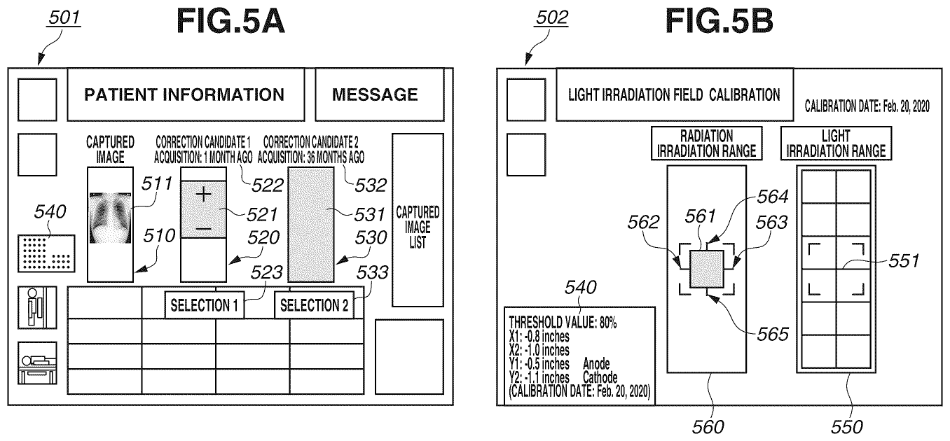

At the time of performing radiographic imaging at, for example, a hospital or clinic, in a case where, for example, in step S204 illustrated in FIG. 2B, there is a plurality of gain correction images, it is necessary to perform image capturing in an image region for which gain correction is feasible. Therefore, first, in step S301, the radiation imaging system 1000 displays a gain correction feasible region on the display unit 1170. While an example of a screen in which to display the gain correction feasible region is described below with reference to, for example, FIGS. 5A and 5B, it is desirable that the screen be compatible with the large-sized radiation imaging unit 1030 in an actual real space.

Next, in step S302, the gain correction data selection unit 1120 selects a gain correction image to be used from among a plurality of gain correction images based on, for example, an operation input to the operation panel 1160. Here, the gain correction data selection unit 1120 selects a gain correction image based on, for example, whether the gain correction image has been acquired from the gain correction feasible region, whether the gain correction image is the newest acquired image, or whether the gain correction image is the one acquired in the same installation environment. Since, usually, not moving the radiation generation unit 1010 or the radiation imaging unit 1030 to a great extent is frequent, step S301 and step S302 can be omitted. Thus, the setting of using a gain correction image which has been used in the previous image capturing can be employed.

Next, in step S303, the radiation imaging system 1000 performs radiographic imaging of the subject H. The user such as a medical worker places the subject H at a position available for radiographic imaging, moves the radiation generation unit 1010 in such a way as to enable image capturing of a region of interest of the subject H, and narrows down a range for radiating radiation R in the radiation diaphragm 1020. On this occasion, it is desirable to perform movement in such a way as to enable image capturing of a region of interest of the subject H within a range of the gain correction feasible region in step S301 and step S302. After the irradiation field of radiation R generated by the radiation generation unit 1010 under a radiation generation condition set via the operation panel 1160 is narrowed down by the radiation diaphragm 1020, radiographic imaging of a region of interest of the subject H is performed. Then, the radiation imaging unit 1030 captures a radiographic image based on incident radiation R (also including radiation R having penetrated the subject H).

Next, in step S304, the image correction unit 1090 performs image processing such as performing gain correction of the radiographic image captured in step S303 with use of the gain correction image selected in step S302. Here, the image processing to be performed includes, for example, pre-processing (dark current correction processing, gain correction processing, and lost pixel correction processing) and post-processing (gradation processing and frequency processing).

Next, in step S305, the display unit 1170 performs image display for confirming the radiographic image (captured image) subjected to image processing in step S304.

Next, in step S306, for example, the CPU 1140 or the gain correction determination unit 1130 determines whether the currently selected gain correction image is OK based on, for example, an operation input to the operation panel 1160.

If, in step S306, it is determined that the currently selected gain correction image is not OK (is NG) (NO in step S306), the CPU 1140 or the gain correction determination unit 1130 advances the processing to step S307. In step S307, the radiation imaging system 1000 displays the gain correction feasible region on the display unit 1170 again.

Next, in step S308, the gain correction data selection unit 1120 selects a gain correction image to be used (a gain correction image different from the previous one) from among the plurality of gain correction images again based on, for example, an operation input to the operation panel 1160. After that, the radiation imaging system 1000 returns the processing to step S304, thus performing processing using the gain correction image selected in step S308. Here, in a case where there is no gain correction image having an appropriate radiation irradiation range for correction, since, in step S108 illustrated in FIG. 2A, the gain correction image for the time of factory shipment has been stored in, for example, the second gain correction data storing unit 1052, the gain correction data selection unit 1120 can select the stored gain correction image. In this way, it is desirable to retain at least one gain correction image which covers the whole area of the radiation imaging unit 1030 as a radiation irradiation range.

If, in step S306, it is determined that the currently selected gain correction image is OK (YES in step S306), the CPU 1140 or the gain correction determination unit 1130 advances the processing to step S309. In step S309, the radiation imaging system 1000 stores the captured radiographic image.

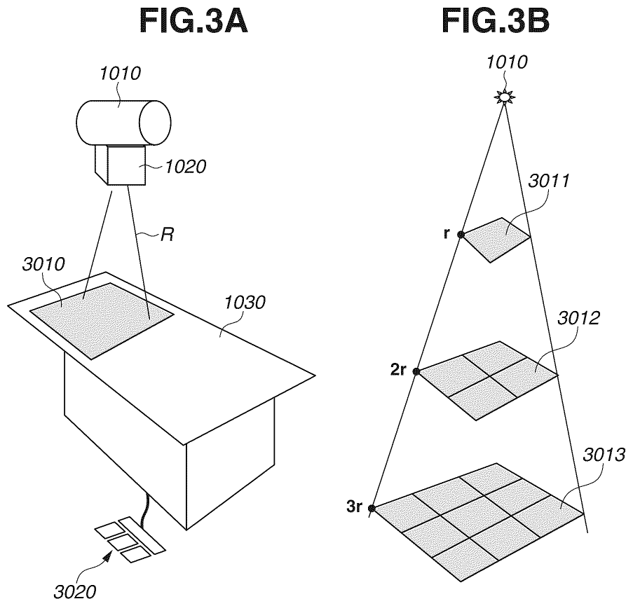

FIGS. 3A and 3B are diagrams used to explain a radiation irradiation range (radiation irradiation field), which is an irradiation range of radiation R, in the radiation imaging unit 1030 according to the first exemplary embodiment. In FIGS. 3A and 3B, constituent elements similar to the constituent elements illustrated in FIG. 1 are assigned the respective same reference characters as those illustrated in FIG. 1, and the detailed description thereof is omitted here.

In FIG. 3A, the radiation generation unit 1010, the radiation diaphragm 1020, and the radiation imaging unit 1030 are illustrated. FIG. 3A illustrates an example in which the radiation imaging unit 1030 is mounted on a recumbent table. In FIG. 3A, a radiation irradiation range (radiation irradiation field) 3010, which is an irradiation range of radiation R radiated to the radiation imaging unit 1030 via the radiation generation unit 1010 and the radiation diaphragm 1020, is illustrated. Additionally, in FIG. 3A, a foot switch 3020, which is operable to increase and decrease the height of the recumbent table with the radiation imaging unit 1030 mounted thereto, is also illustrated.

If the length of the long side of an effective pixel range of the radiation imaging unit 1030 becomes larger than or equal to 18 inches, since the weight thereof becomes larger according to the size of the effective pixel range, the radiation imaging unit 1030 becomes difficult to handle and thus becomes unable to be readily moved. The user such as a medical worker moves the radiation generation unit 1010, which is suspended from a ceiling, to a position at which the radiation generation unit 1010 is able to radiate radiation R to the subject H. Radiation R generated by the radiation generation unit 1010 is radiated to the large-sized radiation imaging unit 1030 while being narrowed down in irradiation field by the radiation diaphragm 1020. With regard to the radiation diaphragm 1020, according to “JIS Z 4712:1998 Guide” for diagnostic X-ray movable diaphragms, the maximum X-ray irradiation field is defined as “in SID 65 cm, do not exceed 35 cm×35 cm” (“SID” denoting a distance between an X-ray tube focus and an image reception area and “cm” denoting centimeter). The area of the radiation irradiation range (radiation irradiation field) 3010 is proportional to the square of the distance from the radiation generation unit 1010.

FIG. 3B is a diagram illustrating the manner in which the areas of radiation irradiation ranges (radiation irradiation fields) 3011, 3012, and 3013 become larger in proportion the respective squares of the distances (r, 2r, and 3r). Assuming that the area of the radiation irradiation range (radiation irradiation field) 3011 in the case of the image capturing distance r=65 cm is 35 cm×35 cm, the area of the radiation irradiation range (radiation irradiation field) 3012 in the case of the image capturing distance 2r=130 cm becomes 70 cm×70 cm. Similarly, the area of the radiation irradiation range (radiation irradiation field) 3013 in the case of the image capturing distance 3r=195 cm becomes 105 cm×105 cm.

The standards of sizes in conventional X-ray films are, for example, as follows:
Ohban (Large Size): 43.2 cm×43.2 cm (17 inches×17 inches),
Hansetsu (Half-cut): 35.6 cm×43.2 cm (14 inches×17 inches),
Daikaku (Big Square): 35.6 cm×35.6 cm (14 inches×14 inches), and
Yotsugiri (Quarter): 25.4 cm×30.5 cm (10 inches×12 inches).

Therefore, if the size of the radiation imaging unit 1030 is 43.2 cm (17 inches) or less, as long as the image capturing distance is set to 80 cm or more, it is possible to avoid the situation in which it is not possible to radiate radiation R to the whole area of the radiation imaging unit 1030. Even with regard to a radiation imaging unit using semiconductors, to make the radiation imaging unit insertable into a conventional upright stand or recumbent table and usable without changing of the usability of, for example, a radiographer, the radiation imaging unit has been frequently designed with approximately the same size as that of an X-ray film. In a case where it is necessary to perform image capturing with a larger size, image capturing can be performed while a plurality of radiation imaging units is made to overlap each other or while a radiation imaging unit and a radiation generation unit are moved for each step. However, in the case of a size of 43.2 cm×86.4 cm (17 inches×34 inches), which corresponds to two radiation imaging units, the image capturing distance needs to be 160 cm or more. Additionally, in the case of a size of 43.2 cm×129.6 cm (17 inches×51 inches), which corresponds to three radiation imaging units, the image capturing distance needs to be 240 cm or more.

Recently, in semiconductor manufacturing equipment, it has become possible to manufacture a large flat panel and it has also become possible to manufacture a radiation imaging unit having a size equivalent to two areas or three areas of Hansetsu (Half-cut) Size. If tiling in which these flat panels are arranged side by side in the horizontal direction enables performing tiling without any gaps on a pixel-by-pixel basis, for example, it becomes possible to set the whole wall or whole floor as a radiation imaging unit.

Radiation diaphragms (X-ray diaphragms) are regulated by, for example, “JIS Z 4712:1998 Guide” in such a manner that radiation (X-rays) is narrowed down in such a way as to be prevented from being radiated. While, here, an example in Japanese Industrial Standards (JIS) has been described, the narrowing-down range may somewhat vary depending on countries. In a case where, to acquire a radiographic image by one round of radiographic imaging using a plurality of radiation imaging units, the plurality of radiation imaging units is mounted on an upright stand, if image capturing is performed with the image capturing distance set to, for example, 250 cm, it has been possible to perform gain correction. However, in a case where the plurality of radiation imaging units is mounted on a recumbent table or in a case where the whole wall is set as a radiation imaging unit, some measures are necessary.

On the other hand, the critical size of a radiation diaphragm (X-ray diaphragm) is very small, and, when the image capturing distance is 100 cm, the length of the long side thereof is about 53.8 cm (the length of the long side being 21.2 inches and the length of the diagonal being 29.9 inches). When the image capturing distance is 110 cm, the length of the long side thereof is about 59.2 cm (the length of the long side being 23.3 inches and the length of the diagonal being 32.9 inches). If a radiation imaging unit with a size less than or equal to these sizes is used, since it is difficult to perform image capturing by one round of radiation irradiation, joints may occur.

Calibration can be classified into a calibration using radiation R and a calibration not using radiation R. Examples of the calibration using radiation R include gain correction and image lag correction. The gain correction includes preliminarily radiating radiation R and then obtaining correction data (for example, a gain correction image), to correct a difference in gain for every pixel of the radiation imaging unit 1030 or a sensitivity distribution of a phosphor thereof. The image lag correction includes, to correct an image lag which differs according to models of the radiation imaging unit 1030, preliminarily radiating radiation R to the whole area and then obtaining correction data based on a time required until radiation R is radiated next and the amount of radiation R remaining in an image. Examples of the calibration not using radiation R include dark current correction for correcting variation in dark current for every pixel of the radiation imaging unit 1030.

The calibration using radiation R has an issue in which, if the size of the radiation imaging unit 1030 exceeds a predetermined size, in a case where, in a hospital or clinic being an installation location, it is impossible to radiate radiation R to the whole area of the radiation imaging unit 1030, joints of irradiation may occur. Particularly, the gain correction has to be performed for all of the pixels of the radiation imaging unit 1030. Although the radiation imaging unit 1030 using semiconductors is manufactured in such a manner that characteristics of the respective pixels are made appropriately uniform, the characteristics are still subtly different from each other for the respective pixels. The gain correction includes correcting such un-uniformity of all of the pixels (about ten million pixels) of the radiation imaging unit 1030.

### The Basic Correction Processing is as Follows:

Corr(x, y)=[Orig(x, y)−Offset(x, y)]/White(x, y), and
White(x, y)=>(n=1 to N) [White(x, y)n-Offset(x, y)n]/N.
   Here, Corr(x, y) denotes a corrected image (an image subjected to correction). Orig(x, y) denotes an uncorrected image (an image not yet subjected to correction). Offset(x, y) denotes an offset correction image. White(x, y) denotes a gain correction image.

The method of using a flat image without any subject H and outputting such an image in a uniform manner is the gain correction. The gain correction image is obtained by irradiation of radiation R and, therefore, cannot be said to be completely uniform. Un-uniformity can be broadly divided into two elements. The first element is shading caused by, for example, a tube heel effect of the radiation generation unit. The shading is a pattern of a low spatial frequency in which intensity gently changes. The second element is radiation quantum noise. On a radiographic image, quantum noise caused by radiation quanta is inevitably superposed. Since the signal-to-noise (S/N) ratio of the radiographic image is proportional to the square root of a dosage and, therefore, can be improved by increasing the dosage. The gain correction is performed on the assumption that these ununiform radiographic images are uniform. This is because, since the un-uniformity of the radiation imaging unit has also bilateral characteristics, i.e., shading and variation for every pixel, whether the un-uniformity observed at the time point of capturing a gain correction image is derived from a radiographic image or is derived from a radiation imaging unit is not determinable. In a case where the heel effect is the cause, if the tube of the radiation generation unit is used vertically upside down, the heel effect results in being doubled. While there is no problem at all with an upright stand, a recumbent table requires attention.

While, depending on users, there are facilities in which daily calibration, including the meaning of daily checking, is being performed, with regard to the radiation imaging unit 1030 with a large effective pixel range (effective image capturing area), there is a case where it is not possible to radiate radiation to the whole effective pixel range at one time. Therefore, the gain correction is appropriately performed with use of a method to which the present disclosure can be applied. Since there is also a possibility of, for example, a change in surrounding environment or a replacement of the radiation generation unit 1010 occurring, in a hospital or clinic, it is necessary to incorporate such measures as to be able to acquire a gain correction image into the radiation imaging system 1000. In the case of the radiation imaging system 1000 of the upright type, it has been possible to set the distance of the radiation generation unit 1010 to 2 meters (m) or more. However, in the case of the radiation imaging system 1000 of the recumbent type, in view of the height of a ceiling being limited and the height of a recumbent bed requiring 80 cm to 100 cm, it is often impossible to set the distance to 1.5 m (150 cm) or more. In a case where the image capturing distance is 150 cm, with regard to the effective pixel range, the length of the long side thereof being about 80.8 cm (the length of the long side being 31.8 inches and the length of the diagonal being 44.8 inches) becomes a critical size. With regard to the radiation imaging unit 1030 the effective pixel range of which is larger than the above-mentioned size, in the placement of a commonly used recumbent apparatus, it becomes difficult to perform, for example, periodic calibration.

Even in a usage environment of the radiation imaging unit 1030, there occur a radiation irradiation region the frequency of which becomes higher as the effective pixel range of the radiation imaging unit 1030 becomes larger and a radiation irradiation region the frequency of which is low, so that a difference may occur in cumulative radiation irradiation amount. Although depending on a phosphor, which is a material for converting radiation (X-rays) R into light, for example, in a case where cesium iodide (CsI) is used as a phosphor, there is the possibility that, as a result of repeated use over the years, due to a difference in cumulative radiation irradiation amount, a dissimilarity in granularity or a gain difference occurs in a radiographic image. By performing, for example, returning the radiation imaging unit 1030 to a factory, it is possible to prepare whole-area gain correction. However, in, for example, a small hospital or clinic, image capturing accompanied by radiation irradiation may not be able to be performed on the whole area of the radiation imaging unit 1030. In such a case, in each hospital or clinic, the need of applying gain correction to only some regions of the radiation imaging unit 1030 to radiate radiation R may arise.

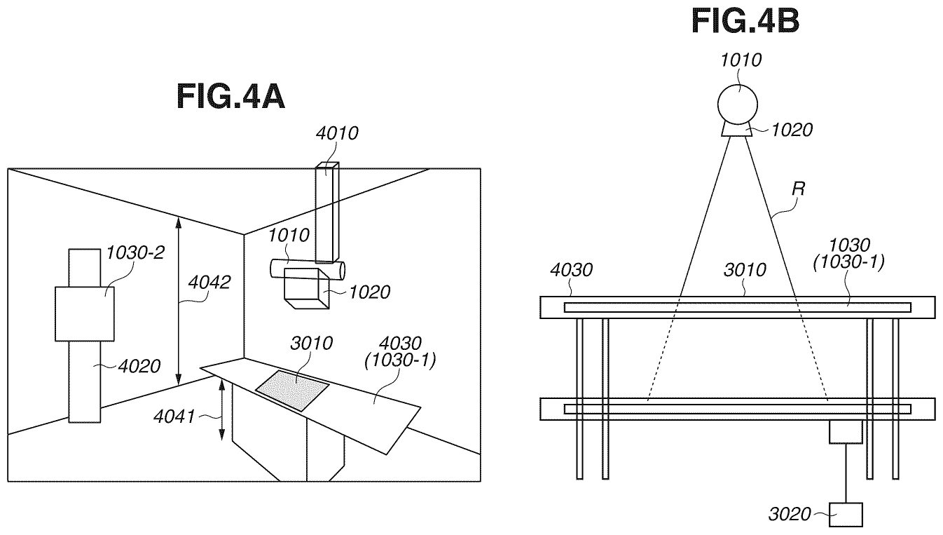

FIGS. 4A and 4B are diagrams illustrating an example of installation of the radiation imaging unit 1030 according to the first exemplary embodiment.

In FIGS. 4A and 4B, constituent elements similar to the constituent elements illustrated in FIG. 1 and FIGS. 3A and 3B are assigned the respective same reference characters as those illustrated in FIG. 1 and FIGS. 3A and 3B, and the detailed description thereof is omitted here.

In the example illustrated in FIG. 4A, the radiation generation unit 1010 is suspended by a stanchion 4010 from a ceiling, so that changing of the height thereof and changing of the position thereof can be performed inside a radiation room. In the example illustrated in FIG. 4A, the radiation generation unit 1010 is equipped with the radiation diaphragm 1020, so that, with respect to radiations generated in all directions, radiations outside of a predetermined range can be blocked. A radiation imaging unit 1030-2 is a radiation imaging unit for upright image capturing. The radiation imaging unit 1030-2 is supported by an upright stand 4020, and is equipped with, for example, an upward and downward movement mechanism and a mechanism which a subject grasps by hand in such a way as to reduce the amount of movement of the subject during image capturing. In the first exemplary embodiment, incorporating the radiation imaging unit 1030-2 into the upright stand 4020 enables, without work of upward and downward movement of the radiation imaging unit 1030-2, radiating radiation R to the subject H to perform image capturing.

Inside the same radiation room, a recumbent top plate 4030, which is available for recumbent image capturing, is arranged. As with the upright stand 4020, it is possible to incorporate a radiation imaging unit 1030-1 with a size of 18 inches or more, to which the present disclosure can be applied, into the recumbent top plate 4030. In addition to FIG. 4A, FIG. 4B also illustrates such a configuration. Only moving the radiation generation unit 1010 enables, without horizontally moving the radiation imaging unit 1030-1 or the recumbent top plate 4030, setting the radiation irradiation range (radiation irradiation field) 3010 to perform image capturing of the subject H. The recumbent top plate 4030 or the subject H not moving during examination has an advantage in safely performing examination. For example, if the recumbent top plate 4030 or the subject H accidentally moves during an examination accompanied by puncture, there occurs a case where it is necessary to take safety into consideration, or there occurs a case where attention needs to be paid in such a way as to prevent, for example, the cord of a fiberscope serving as an endoscope from being stuck.

The height 4041 of the recumbent top plate 4030 illustrated in FIG. 4A is set to, for example, 70 cm, which is the standard height of a bed. To put the subject H on the recumbent top plate 4030, the recumbent top plate 4030 can be vertically moved to set the height thereof to 30 cm to 40 cm. The foot switch 3020 illustrated in FIG. 4B for adjusting the height 4041 of the recumbent top plate 4030 is configured to be able to be controlled without the use of both hands, and the recumbent top plate 4030 is configured to, when the height 4041 thereof is vertically adjusted, stop at the preliminarily set standard height of 70 cm once.

The user such as a medical worker measures distance by a stanchion scale present on the stanchion 4010 or a tape-type distance measurement unit or laser-type distance measurement unit belonging to the radiation diaphragm 1020 or the radiation generation unit 1010, and frequently sets the image capturing distance to 90 cm to 130 cm in normal cases. The height 4042 of the ceiling is determined based on an architectural unit of measurement for each method of construction, for example, from among 240 cm to 280 cm.

If the image capturing distance is set to 130 cm relative to the height 4041 of the recumbent top plate 4030 being 70 cm, the height of the radiation generation unit 1010 becomes 200 cm and the radiation generation unit 1010 collides with the ceiling suspender due to the size of the radiation generation unit 1010, so that there may be a limit to increasing the distance by raising the radiation generation unit 1010. It can become difficult for, for example, some workers to adjust the height of the radiation generation unit 1010 to 200 cm. Therefore, restrictions arise to set the image capturing distance to about 90 cm to 120 cm, so that many medical facilities set the image capturing distance to about 100 cm in recumbent image capturing.

The maximum X-ray irradiation field (radiation irradiation range) satisfying “JIS Z 4712:1998 Guide” is as follows. With regard to the size being about 48.4 cm (about 19 inches) or more and the diagonal being 27 inches or more when the image capturing distance is 90 cm and the size being about 53.8 cm (about 21 inches) or more and the diagonal being 30 inches or more when the image capturing distance is 100 cm, it is impossible to perform X-ray irradiation (radiation irradiation). Therefore, in a case where the radiation imaging unit 1030 with a size exceeding the above-mentioned sizes is used in a recumbent position, it is impossible to perform image capturing with radiation R radiated to the whole effective pixel range. In a case where the large-sized radiation imaging unit 1030 the length of the short side of which exceeds 19 inches (27 inches in the length of the diagonal) is used in a recumbent position, as mentioned above, there are great advantages in, for example, safety, reduction of the image capturing preparation time, and cutdown of a horizontal movement mechanism.

On the other hand, as the size of the radiation imaging unit 1030 becomes larger, a division may be made into a region to which radiation R is frequently radiated and a region in which this is not the case. A semiconductor or phosphor inside the radiation imaging unit 1030, after being used for many years depending on the cumulative amount of radiation, may cause a difference in sensitivity or noise. In an early stage after installation, a gain correction image acquired in the flowchart (SS_A1) for the time of factory shipment illustrated in FIG. 2A is able to be used. However, a change in characteristics accompanied by the many year's cumulative radiation amount may take place. Therefore, it is desirable to use a gain correction image acquired as recently as possible.

With the matter being reduced to the upright stand 4020, while it has been possible to overlap a plurality of conventional radiation imaging units and then perform image capturing only in a determined geometric placement, restricting image capturing to that only in a determined geometric placement is substantially inconvenient in many cases. For example, the method of returning the radiation imaging unit to a factory once and then acquiring a gain correction image can also be taken as an example of measure, but may be substantially difficult with respect to, for example, a remote location.

In the radiation imaging unit 1030 to which the present disclosure can be applied, it is possible to, in a region to which radiation R is radiated, acquire gain correction images in a plurality of radiation irradiation ranges and, at the time of acquiring each gain correction image, select an appropriate gain correction image with use of radiation irradiation range information. For example, the present disclosure can also be applied to, for example, a flat panel detector (FPD) (radiation imaging unit 1030) set to the whole wall or an FPD (radiation imaging unit 1030) set to the whole bed.

FIGS. 5A and 5B are diagrams illustrating examples of screens each of which can be displayed on the display unit 1170 in the radiation imaging system 1000 according to the first exemplary embodiment.

The screen 501 illustrated in FIG. 5A is a screen for use in acquiring a radiation irradiation range, and has the functions of the display unit 1170 and the operation panel 1160. In the screen 501, a radiographic image 510, which is a captured image, a first correction image (in the first exemplary embodiment, a first gain correction image) 520, a second correction image (in the first exemplary embodiment, a second gain correction image) 530 are displayed.

The outer frame of each of the radiographic image 510, the first correction image 520, and the second correction image 530 represents, for example, the whole area of an entrance surface on which radiation R is incident of the radiation imaging unit 1030.

In the radiographic image 510, which is a captured image, a radiographic image region of the subject H is a radiation irradiation range 511 for image capturing.

In the first correction image 520, a grayish region is a radiation irradiation range 521 for correction. In the vicinity of the first correction image 520, information (in the example illustrated in FIG. 5A, information about the acquisition moment) 522 about the first correction image 520 and a “selection 1” button 523, which is operated to select the first correction image 520, are displayed.

In the second correction image 530, a grayish region is a radiation irradiation range 531 for correction. In the vicinity of the second correction image 530, information (in the example illustrated in FIG. 5A, information about the acquisition moment) 532 about the second correction image 530 and a “selection 2” button 533, which is operated to select the second correction image 530, are displayed.

Here, while, in the example illustrated in FIG. 5A, the information 522 about the first correction image 520 and the information 532 about the second correction image 530 denote pieces of information about the respective acquisition moments of the first correction image 520 and the second correction image 530, the first exemplary embodiment is not limited to this. For example, information indicating in which radiation room the correction image has been acquired and information indicating in which of the upright position and the recumbent position the correction image has been acquired can also be additionally displayed. The, the second correction image 530 can be set as a correction image serving as a runner-up (for example, a runner-up in use frequency) candidate for the first correction image 520. As the first correction image 520 or the second correction image 530, a gain correction image stored at the time of factory shipment in the flowchart illustrated in FIG. 2A can be displayed. Since there is a limit to the number of images that are able to be displayed in the screen 501, it is desirable to preliminarily set the display sequence of gain correction images serving as candidates.

In an example of the screen 501 illustrated in FIG. 5A, the first correction image 520, which is a recently acquired correction image, is displayed together with the radiation irradiation range 521 for correction therefor. In a case where another gain correction is acquired, the second correction image 530, which is a correction image second close to the present time, is displayed together with the radiation irradiation range 531 for correction therefor. In the example illustrated in FIG. 5A, in view of the information 532 about the acquisition moment, the second correction image 530 is, for example, a correction image acquired 36 months before factory shipment.

In the example illustrated in FIG. 5A, a partial range of the radiation irradiation range 511 for image capturing (a lower range of the radiation irradiation range 511 for image capturing) in the radiographic image 510, which is a captured image, is not contained in the radiation irradiation range 521 for correction in the first correction image 520. In this situation, in a case where the user such as a medical worker has operated the “selection 1” button 523 to select the first correction image 520 as a correction image for correcting the radiographic image 510, it is not possible to perform appropriate correction (in the first exemplary embodiment, gain correction) to the radiographic image 510. On the other hand, in the example illustrated in FIG. 5A, the entire range of the radiation irradiation range 511 for image capturing in the radiographic image 510 is contained in the radiation irradiation range 531 for correction in the second correction image 530. In this situation, in a case where the user such as a medical worker has operated the “selection 2” button 533 to select the second correction image 530 as a correction image for correcting the radiographic image 510, it is possible to perform appropriate correction (in the first exemplary embodiment, gain correction) to the radiographic image 510. In the first exemplary embodiment, the display unit 1170 displays, in a selectable manner, at least one correction image 530 available for appropriate correction from a plurality of correction images 520 and 530 different in irradiation condition of radiation R, based on the radiation irradiation range 511 for image capturing in the radiographic image 510. While, in the example illustrated in FIG. 5A, one correction image 530 is displayed as a correction image having a radiation irradiation range for correction containing the entire radiation irradiation range 511 for image capturing in the radiographic image 510, the first exemplary embodiment can be configured to display two or more such correction images. In the first exemplary embodiment, the display unit 1170 displays, in a comparable manner, the radiation irradiation range 511 for image capturing and a plurality of radiation irradiation ranges 521 and 531 for correction in a plurality of correction images 520 and 530. This enables the user to select at least one correction image 530 available for appropriate correction from among a plurality of correction images 520 and 530 by visually recognizing the radiation irradiation range 511 for image capturing and the plurality of radiation irradiation ranges 521 and 531 for correction, which are displayed for comparison.

In the first exemplary embodiment, the radiation irradiation range 511 for image capturing can be acquired by performing image analysis of the radiographic image 510. Similarly, in the first exemplary embodiment, the radiation irradiation range 521 for correction can be acquired by performing image analysis of the first correction image 520, and, moreover, the radiation irradiation range 531 for correction can be acquired by performing image analysis of the second correction image 530. The threshold value to be used for this image analysis can be set or changed by the user via the operation panel 1160. The acquisition method for a radiation irradiation range can also be a method other than the above-mentioned image analysis, i.e., for example, a method in which the user performs an operation to input four points indicating the boundary of a radiation irradiation range on the screen 501. The above-mentioned radiation irradiation range can be acquired by using one or more pieces of information selected from information about a focus position of the radiation generation unit, information about a relative position, information about a relative angle, and information about the image capturing distance between the radiation generation unit and the radiation imaging unit, and information about the aperture opening degree of the radiation diaphragm.

In the screen 501 illustrated in FIG. 5A, the user determines whether to perform an operation to select the “selection 1” button 523 for the first correction image 520 or whether to perform an operation to select the “selection 2” button 533 for the second correction image 530, based on the radiation irradiation range 511 for image capturing in the radiographic image 510. To assist the user in performing such determination, the display unit 1170 can display, in a correction image, information about at least one of the anode (+) or cathode (−) of the radiation generation unit 1010. In the screen 501 illustrated in FIG. 5A, information about both the anode (+) and cathode (−) of the radiation generation unit 1010 is displayed in the first correction image 520.

Then, the image correction unit 1090 corrects (in the first exemplary embodiment, performs gain correction of) the radiographic image 510 with use of a correction image (for example, the second correction image 530) selected from at least one correction image displayed by the display unit 1170 in a selectable manner.

The screen 502 illustrated in FIG. 5B is a screen that is displayed on the display unit 1170 to be used to calibrate a light irradiation range (light irradiation field), which is an irradiation range of light emitted from the light source of the radiation diaphragm 1020. The calibration of the above-mentioned light irradiation range (light irradiation field) is periodically performed in advance. The radiation diaphragm 1020 varies by medical facility, and, in some cases, is adjusted by a service engineer for the radiation generation unit 1010 in medical practice. In the first exemplary embodiment, it is desirable that the above-mentioned light irradiation range (light irradiation field) be being periodically calibrated in advance. In the screen 502 illustrated in FIG. 5B, an exterior image 550 of the radiation imaging unit 1030 and a light irradiation field calibration image 560 are displayed. In the case of the screen 502 illustrated in FIG. 5B, first, the display indicating “Please fit the light irradiation field to this range to perform image capturing.” appears. A target light irradiation range 551 is indicated together with the exterior image 550 of the radiation imaging unit 1030. Then, a result obtained by performing image capturing while fitting the light irradiation field to the target light irradiation range 551 is displayed in the light irradiation field calibration image 560. In the light irradiation field calibration image 560, the actual irradiation range of radiation R is illustrated as a radiation irradiation range 561. Then, the difference between the target light irradiation range 551 and the actual radiation irradiation range 561 is displayed as a difference 562 on the left side and a difference 563 on the right side in the X-axis and a difference 564 on the upper side and a difference 565 on the lower side in the Y-axis. Then, a result obtained by calibrating the difference between the radiation irradiation range and the light irradiation range is displayed, together with the year, month, and day of calibration, in a light irradiation range calibration result display region 540 illustrated in each of FIG. 5A and FIG. 5B. While, in FIGS. 5A and 5B, the radiation irradiation range is illustrated on the presumption that the radiation irradiation range is quadrilateral (rectangular), the first exemplary embodiment is not limited to the radiation irradiation range being quadrilateral (rectangular), but can include the radiation irradiation range being polygonal or circular.

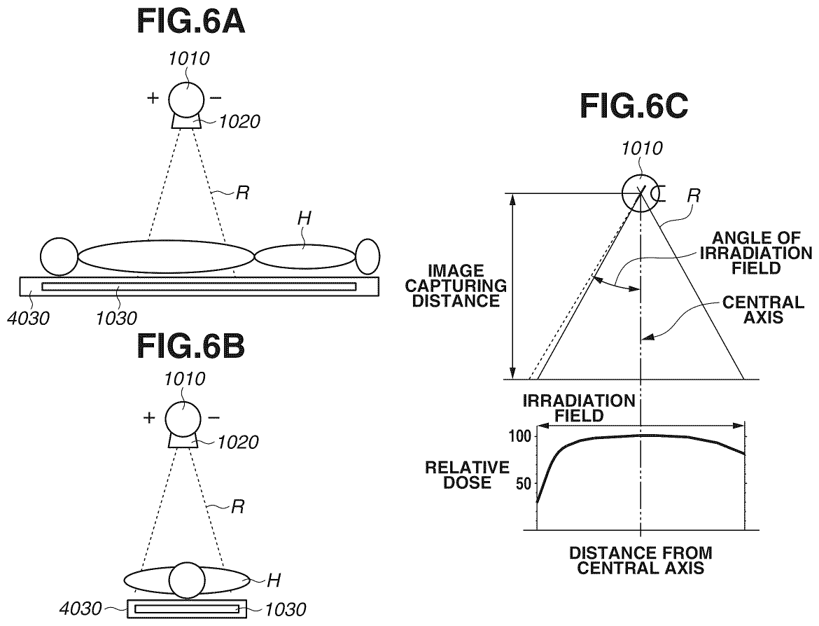

FIGS. 6A, 6B, and 6C are diagrams used to explain an example of an application in which it is supposed to increase the image quality by displaying not only information about a radiation irradiation range but also ancillary information in the radiation imaging system 1000 according to the first exemplary embodiment. For example, an example of an application in which it is supposed to more increase the image quality by also displaying radiation irradiation distribution information included in the radiation irradiation range is explained. In FIGS. 6A, 6B, and 6C, constituent elements similar to the constituent elements illustrated in FIG. 1, FIGS. 3A and 3B, and FIGS. 4A and 4B are assigned the respective same reference characters as those illustrated in FIG. 1, FIGS. 3A and 3B, and FIGS. 4A and 4B, and the detailed description thereof is omitted here.

In FIG. 6A and FIG. 6B, the directions of the anode (+) and cathode (−) of the radiation generation unit 1010 relative to the subject H and the radiation imaging unit 1030 are illustrated. In FIG. 6C, a relationship between the image capturing distance between the radiation generation unit 1010 and the radiation imaging unit 1030 and the irradiation field and a relationship between the relative dose of radiation R and the distance from the central axis are illustrated.

As the radiation imaging unit 1030 becomes larger, a radiation irradiation distribution becomes more likely to occur. The cause of the radiation irradiation distribution occurring is, for example, a difference in length from the radiation generation unit 1010 to the inside of the radiation imaging unit 1030, a difference between the direction parallel to and the direction perpendicular to the anode and cathode of the radiation generation unit 1010, and an attenuation distribution or scattering radiation distribution inside the radiation diaphragm 1020 or the radiation imaging unit 1030.

Without performing determination by the specifications of the radiation generation unit 1010 and the radiation imaging unit 1030, it is necessary to consider an image capturing purpose, and, particularly, whether to perform diagnostic imaging using a symmetric property of the human body. There are roughly two patterns, i.e., a case where the anode and cathode of the radiation generation unit 1010 are set in the longitudinal direction relative to the human body and a case where the anode and cathode of the radiation generation unit 1010 are set in the transverse direction relative to the human body. The first pattern is a case where it is effective to diagnose an image on the premise of a symmetric property of the human body. In such a case, it is desirable to set the direction of the anode and cathode of the radiation generation unit 1010 to the longitudinal direction. The reason for this is that, if the direction of the anode and cathode of the radiation generation unit 1010 is set to the transverse direction, the variance of radiation irradiation of the anode and cathode in the transverse direction may look like, for example, a density difference between right and left lungs, thus leading to an erroneous diagnosis or a feeling of image strangeness. However, on the minus side, if the direction of the anode and cathode of the radiation generation unit 1010 is set to the longitudinal direction, shading becomes very large. As the radiation imaging unit 1030 becomes larger in the longitudinal direction, shading is more affected. The second pattern is a case where it is undesirable that there is a density difference in the longitudinal direction. For example, in the case of limbs, setting the direction of the anode and cathode of the radiation generation unit 1010 to the transverse direction may enable obtaining a desirable image. In the case of image capturing for obtaining quantitative information in the density direction, such as a digital image processing (DIP) method or bone mineral density (BMD), it is also desirable to set the direction of the anode and cathode of the radiation generation unit 1010 to a redetermined direction at a predetermined position. In the first exemplary embodiment, it is desirable to also display such ancillary information about the anode (+) and cathode (−) of the radiation generation unit 1010 in the screen 501 illustrated in FIG. 5A (specifically, in the first correction image 520 illustrated in FIG. 5A), thus enabling determining a more appropriate correction image.

The above-described radiation imaging system 1000 according to the first exemplary embodiment includes the radiation imaging unit 1030, which captures a radiographic image based on incident radiation R. The radiation imaging system 1000 according to the first exemplary embodiment includes the radiation irradiation range acquisition units 1061 and 1062, which acquire the radiation irradiation range 511 for image capturing, which is an irradiation range of radiation R used when a radiographic image has been captured by the radiation imaging unit 1030. Additionally, the radiation imaging system 1000 according to the first exemplary embodiment includes the display unit 1170, which displays, in a selectable manner, at least one correction image from a plurality of correction images 520 and 530 different in irradiation condition of radiation R, based on the radiation irradiation range 511 for image capturing.

According to such a configuration, even in a case where it is not possible to radiate radiation R to the whole area of the radiation imaging unit 1030, it is possible to appropriately correct a captured radiographic image.

Additionally, the radiation imaging system 1000 according to the first exemplary embodiment includes the radiation irradiation range storing unit 1110, which stores a radiation irradiation range for correction, which is an irradiation range of radiation R used when respective correction images have been acquired by the radiation imaging unit 1030. Then, the display unit 1170 displays, in a selectable manner, at least one correction image from a plurality of correction images 520 and 530, based on the radiation irradiation range 511 for image capturing and a plurality of radiation irradiation ranges 521 and 531 for correction. More specifically, the display unit 1170 displays, in the screen 501 in a comparable manner, the radiation irradiation range 511 for image capturing in the radiation imaging unit 1030 and a plurality of radiation irradiation ranges 521 and 531 for correction in the radiation imaging unit 1030.

According to such a configuration, even in a case where it is not possible to radiate radiation R to the whole area of the radiation imaging unit 1030, it is possible to appropriately correct a captured radiographic image.

Next, a second exemplary embodiment is described. In the description of the second exemplary embodiment described below, particulars in common with those in the above-described first exemplary embodiment are omitted from description, and only particulars different from those in the above-described first exemplary embodiment are described.

The outline configuration of a radiation imaging system according to the second exemplary embodiment is similar to the outline configuration of the radiation imaging system 1000 according to the first exemplary embodiment illustrated in FIG. 1. In the second exemplary embodiment, a configuration of preliminarily recognizing a radiation irradiation range using a camera image captured by the visible light camera 1040 is described.

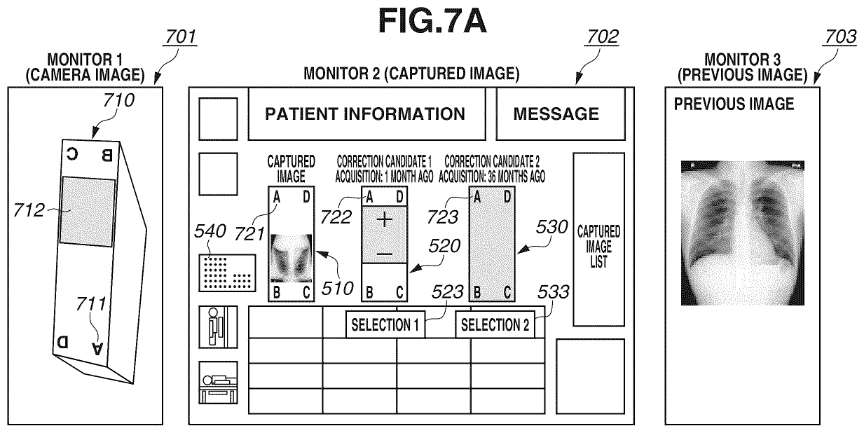

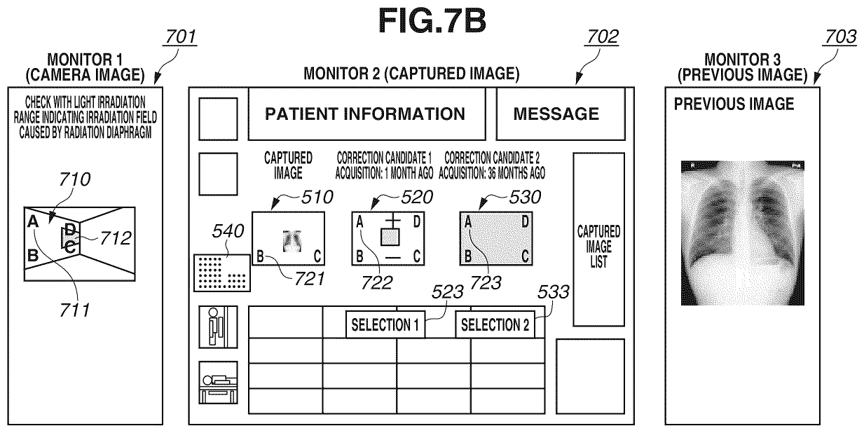

FIGS. 7A and 7B are diagram each illustrating examples of screens that can be displayed on the display unit 1170 in the radiation imaging system 1000 according to the second exemplary embodiment. In FIGS. 7A and 7B, constituent elements similar to the constituent elements illustrated in FIGS. 5A and 5B are assigned the respective same reference characters as those in in FIGS. 5A and 5B, and the detailed description thereof is omitted here.

In FIG. 7A, a screen 701, which displays a camera image captured by the visible light camera 1040, a screen 702, which displays a radiographic image 510 serving as a captured image and, for example, correction images 520 and 530, and a screen 703, which displays the previous captured image (radiographic image), are illustrated. Here, while, in the second exemplary embodiment, the screen 701, the screen 702, and the screen 703 are assumed to be simultaneously displayed on the display unit 1170, the present disclosure includes a configuration of displaying the screen 701, the screen 702, and the screen 703 on the respective different display units (monitors). In the second exemplary embodiment, the screen 702 illustrated in FIG. 7A has the functions of the display unit 1170 and the operation panel 1160.

In the second exemplary embodiment, the large-sized radiation imaging unit 1030 is assumed to be arranged on the whole area of the recumbent table. In the screen 701, a camera image in which a light irradiation range 712 for light emitted from the light source of the radiation diaphragm 1020 and radiated to the radiation imaging unit 1030 is depicted is displayed. The light irradiation range 712 is a range to which light emitted from the light source of the radiation diaphragm 1020, which is arranged in such a way as to be located at the optically same position as the position of focus of radiation R of the radiation generation unit 1010, has been radiated. In the camera image displayed in the screen 701, a radiation imaging unit region 710 equivalent to the radiation imaging unit 1030, in which light emitted from the light source of the radiation diaphragm 1020 has been radiated to the light irradiation range 712, is depicted. In the radiation imaging unit region 710 of the camera image displayed in the screen 701, “A”, “B”, “C”, and “D”, which configure an index 711 indicating the placement position of the radiation imaging unit 1030, are depicted. Similarly, in the radiographic image 510 displayed in the screen 702, “A”, “B”, “C”, and “D”, which configure an index 721 indicating the placement position of the radiation imaging unit 1030, respectively corresponding to the index 711 (“A”, “B”, “C”, and “D”) depicted in the radiation imaging unit region 710, are also depicted. In the first correction image 520 displayed in the screen 702, “A”, “B”, “C”, and “D”, which configure an index 722 indicating the placement position of the radiation imaging unit 1030, respectively corresponding to the index 711 (“A”, “B”, “C”, and “D”) depicted in the radiation imaging unit region 710, are also depicted. In the second correction image 530 displayed in the screen 702, “A”, “B”, “C”, and “D”, which configure an index 723 indicating the placement position of the radiation imaging unit 1030, respectively corresponding to the index 711 (“A”, “B”, “C”, and “D”) depicted in the radiation imaging unit region 710, are also depicted. This enables recognizing the replacement relationship of the radiation imaging unit 1030 in the camera image displayed in the screen 701 and the radiographic image 510, the first correction image 520, and the second correction image 530 displayed in the screen 702. While, in FIG. 7A, “A”, “B”, “C”, and “D” serving as an index, which are shown in the camera image in the screen 701 and the radiographic image 510 and the correction images 520 and 530 in the screen 702, are allocated to the four corners of the radiation imaging unit 1030, the second exemplary embodiment is not limited to this. For example, as long as a mark that uniquely identifies the placement relationship of the radiation imaging unit 1030, such as letters indicating the direction thereof, is used, the mark can be allocated to one corner of the radiation imaging unit 1030. A portion with a product manufacturer name or product name printed thereon can be utilized as an index indicating the placement relationship of the radiation imaging unit 1030.

FIG. 7B illustrates an example of application in which the whole wall is set as a radiation imaging unit region 710 equivalent to the radiation imaging unit 1030 that captures a radiographic image, as depicted in the camera image displayed in the screen 701. In FIG. 7B, constituent elements similar to the constituent elements illustrated in FIG. 7A are assigned the respective same reference characters as those in FIG. 7A, and the detailed description thereof is omitted here. As illustrated in FIG. 7B, when it is possible to capture a radiographic image with the whole wall set as the radiation imaging unit region 710, there is a case where radiation R radiated from the radiation generation unit 1010 is not able to be radiated to the entirety of the whole wall at one time. In such a case, a camera image captured by the visible light camera 1040 mounted to the radiation diaphragm 1020 is displayed in the screen 701. Simultaneously displaying the camera image including the light irradiation range 712 displayed in the screen 701 illustrated in FIG. 7B and the correction images 520 and 530 including radiation irradiation ranges displayed in the screen 702 enables an appropriate correction image to be selected via the “selection 1” button 523 or the “selection 2” button 533.

Next, a processing procedure in a control method for the radiation imaging system 1000 according to the second exemplary embodiment is described. Here, in the control method for the radiation imaging system 1000 according to the second exemplary embodiment, the processing procedure performed at the time of factory shipment is similar to that illustrated in FIG. 2A and the processing procedure performed at the time of installation or at the time of periodic inspection is similar to that illustrated in FIG. 2B, and, therefore, the detailed description thereof is omitted here.

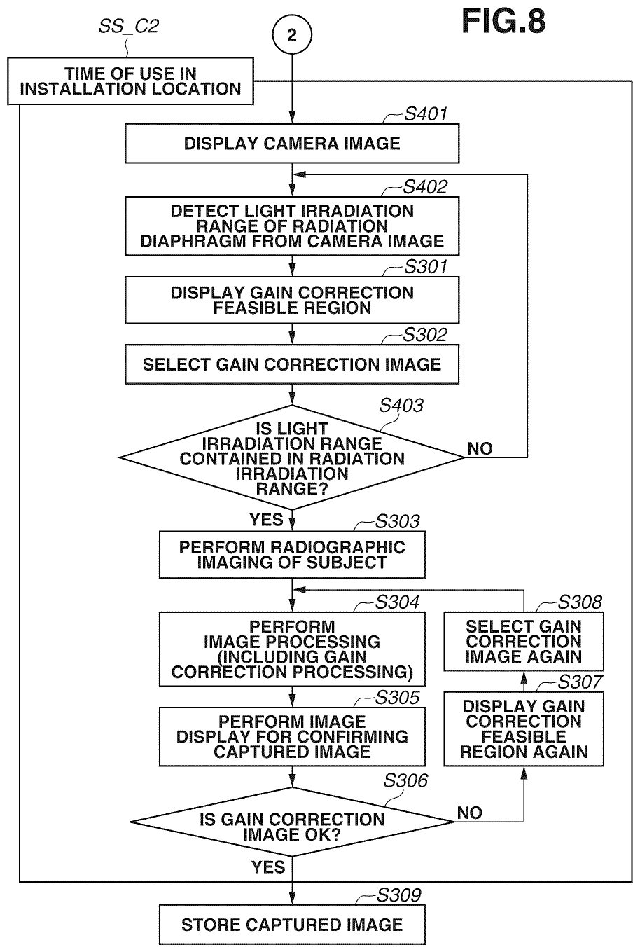

FIG. 8 is a flowchart illustrating an example of a processing procedure performed at the time of use in installation location in the control method for the radiation imaging system 1000 according to the second exemplary embodiment. In FIG. 8, a flowchart illustrating an example of a processing procedure performed at the time of use in installation location in the second exemplary embodiment is denoted by SS_C2. The processing in the flowchart (SS_C2) at the time of use in installation location illustrated in FIG. 8 can be performed after the processing in the flowchart (SS_B1) at the time of installation or at the time of periodic inspection illustrated in FIG. 2B ends. In the processing in the flowchart illustrated in FIG. 8, processing steps similar to those in the processing in the flowchart illustrated in FIG. 2C are assigned the respective same step numbers as those in FIG. 2C, and the detailed description thereof is omitted here.

First, in step S401, the radiation imaging system 1000 displays, in the screen 701 on the display unit 1170, a camera image captured by the visible light camera 1040 mounted to the radiation diaphragm 1020.

Next, in step S402, the radiation imaging system 1000 (for example, the CPU 1140) detects a light irradiation range 712 of light emitted from the light source of the radiation diaphragm 1020, from the camera image acquired in step S401. In the case of specifications in which the light source of the radiation diaphragm 1020 is not an always-on light source but is turned on only when a predetermined button has been pressed, a camera image captured as a still image when the light irradiation range 712 is being illuminated is used. A configuration in which a camera image is captured as a moving image and displaying of the moving image is stopped in the screen 701 with the light irradiation range 712 illuminated therein can be employed.

Next, as with step S301 illustrated in FIG. 2C, the radiation imaging system 1000 displays a gain correction feasible region on the display unit 1170. It is desirable that a gain correction feasible region in the large-sized radiation imaging unit 1030 be displayed in a screen on the display unit 1170.

Next, as with step S302 illustrated in FIG. 2C, the gain correction data selection unit 1120 selects a gain correction image to be used from among a plurality of gain correction images based on, for example, an operation input to the operation panel 1160.

Next, in step S403, the radiation imaging system 1000 determines whether the light irradiation range 712 acquired in step S402 is contained in a radiation irradiation range for correction in the gain correction image selected in step S302. It is taken into consideration that the light irradiation range of light emitted from the light source of the radiation diaphragm 1020 does not necessarily coincide with the radiation irradiation range and may deviate therefrom by about 1 inch. The light irradiation range of light emitted from the light source of the radiation diaphragm 1020 may be set somewhat larger than the actual radiation irradiation range in such a way as to prevent radiation R from being excessively radiated. Therefore, it is desirable to display the screen 502 for calibrating a difference between the radiation irradiation range for correction and the light irradiation range, thus enabling calibrating the light irradiation range via the operation panel 1160.

If, in step S403, it is determined that the light irradiation range 712 acquired in step S402 is not contained in a radiation irradiation range for correction in the gain correction image selected in step S302 (NO in step S403), the radiation imaging system 1000 returns the processing to step S402. Then, in step S402, after the radiation diaphragm 1020 is adjusted, the radiation imaging system 1000 performs processing operations in step S402 and subsequent steps again.

On the other hand, if, in step S403, it is determined that the light irradiation range 712 acquired in step S402 is contained in a radiation irradiation range for correction in the gain correction image selected in step S302 (YES in step S403), the radiation imaging system 1000 advances the processing to step S303. Processing operations in step S303 and subsequent steps are similar to the corresponding processing operations illustrated in FIG. 2C, and, therefore, the detailed description thereof is omitted here.

Next, a third exemplary embodiment is described. In the description of the third exemplary embodiment described below, particulars in common with those in the above-described first and second exemplary embodiments are omitted from description, and only particulars different from those in the above-described first and second exemplary embodiments are described.

The outline configuration of a radiation imaging system according to the third exemplary embodiment is similar to the outline configuration of the radiation imaging system 1000 according to the first exemplary embodiment illustrated in FIG. 1. In the third exemplary embodiment, a configuration of, with use of, for example, a camera image captured by the visible light camera 1040, performing navigation in such a manner that a light irradiation range of light emitted from the light source of the radiation diaphragm 1020 is contained in a radiation irradiation range for a correction image that has been preliminarily set as a selection candidate is described.

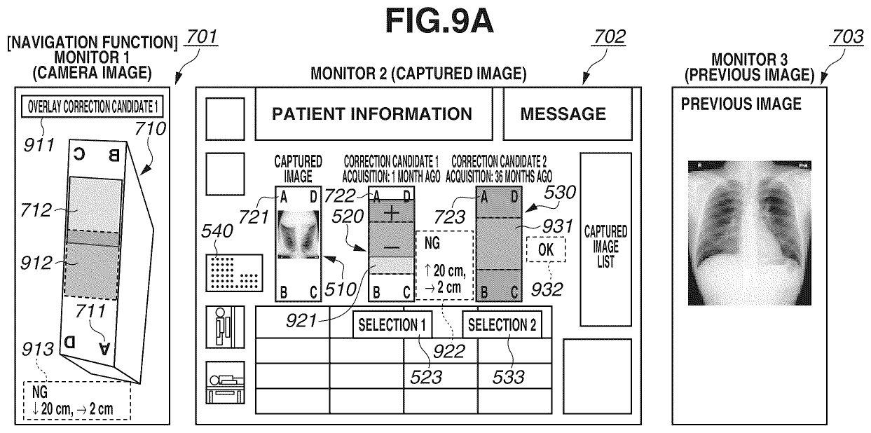

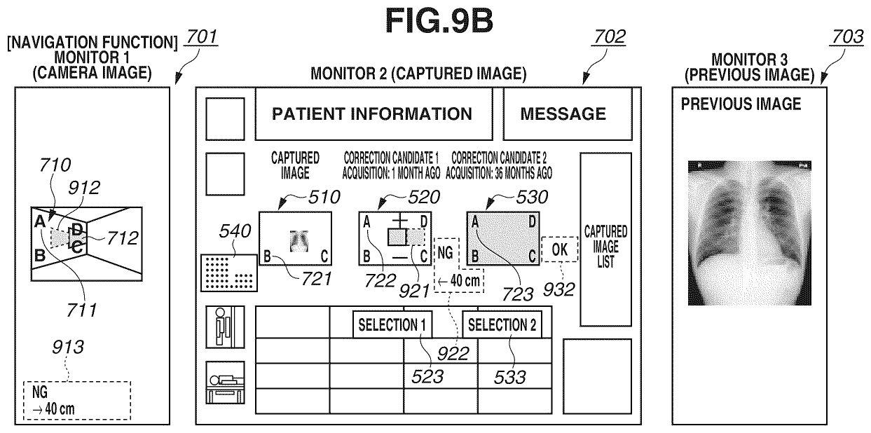

FIGS. 9A and 9B are diagrams each illustrating examples of screens that can be displayed on the display unit 1170 in the radiation imaging system 1000 according to the third exemplary embodiment. In FIGS. 9A and 9B, constituent elements similar to the constituent elements illustrated in FIGS. 5A and 5B and FIGS. 7A and 7B are assigned the respective same reference characters as those in in FIGS. 5A and 5B and FIGS. 7A and 7B, and the detailed description thereof is omitted here.

In FIG. 9A, a screen 701, which displays a camera image captured by the visible light camera 1040, a screen 702, which displays a radiographic image 510 serving as a captured image and, for example, correction images 520 and 530, and a screen 703, which displays the previous captured image (radiographic image), are illustrated. FIG. 9A is a diagram illustrating a screen display example corresponding to FIG. 7A from among FIG. 7A and FIG. 7B. In the example illustrated in FIG. 7A, a camera image in which the light irradiation range 712 is depicted, captured by the visible light camera 1040, is displayed in the screen 701. On the other hand, in the example illustrated in FIG. 9A, in the screen 701, together with the light irradiation range 712, a radiation irradiation range 912 corresponding to the radiation irradiation range (521) of the currently selected first correction image 520 is displayed while being overlaid on the light irradiation range 712.

In the screen 701 illustrated in FIG. 9A, to prevent the currently selected correction image from being confusing, information 911 indicating the currently selected correction image is displayed. The user moves the radiation generation unit 1010 while looking at the screen 701 illustrated in FIG. 9A, thus moving the radiation generation unit 1010 to a position enabling an appropriate camera image to be obtained. In the screen 701 illustrated in FIG. 9A, the amount of movement (deviation amount) of the radiation generation unit 1010 is displayed as navigation information 913. In the navigation information 913, information indicating whether the current position of the light irradiation range 712 is “OK” or “NG” (in FIG. 9A, “NG”) and information indicating the direction and amount of movement to be performed (in FIG. 9A, “↓20 cm, →2 cm”) are included.

In the screen 702 illustrated in FIG. 9A, with regard to the first correction image 520, together with the radiation irradiation range (521) thereof, a light irradiation range 921 corresponding to the light irradiation range 712 displayed in the screen 701 is also displayed while being overlaid on the radiation irradiation range (521). Additionally, as navigation information 922, information indicating whether the current position of the light irradiation range 921 is “OK” or “NG” (in FIG. 9A, “NG”) and information indicating the direction and amount of movement to be performed (in FIG. 9A, “↑20 cm, →2 cm”) are displayed. Similarly, with regard to the second correction image 530, together with the radiation irradiation range (531) thereof, a light irradiation range 931 corresponding to the light irradiation range 712 displayed in the screen 701 is displayed while being overlaid on the radiation irradiation range (531). Additionally, as navigation information 932, information indicating whether the current position of the light irradiation range 931 is “OK” or “NG” (in FIG. 9A, “OK”) is displayed. This enables the user to readily recognize, together with a target radiographic image, a difference between the light irradiation range and the radiation irradiation range for a correction image even on a different axis.

FIG. 9B is a diagram illustrating a screen display example corresponding to FIG. 7B from among FIG. 7A and FIG. 7B. In FIG. 9B, constituent elements similar to the constituent elements illustrated in FIG. 9A are assigned the respective same reference characters as those in FIG. 9A, and the detailed description thereof is omitted here. In a case where, as depicted in a camera image displayed in the screen 701 illustrated in FIG. 9B, the whole wall is set as the radiation imaging unit region 710 equivalent to the radiation imaging unit 1030 capable of capturing a radiographic image, the following may be conceivable. Specifically, the position of radiation R radiated from the radiation generation unit 1010 and the radiation irradiation range for correction which has been used for the previous image may differ from each other. In the example illustrated in FIG. 9A, the visible light camera 1040 mounted to the radiation diaphragm 1020 is used to move the radiation generation unit 1010. On the other hand, in a case where the whole wall is set as the radiation imaging unit region 710 equivalent to the radiation imaging unit 1030 capable of capturing a radiographic image, as illustrated in FIG. 9B, even when being placed at a position enabling capturing an image of the whole area, the visible light camera 1040 can be used as navigation for the radiation irradiation range in the present disclosure.

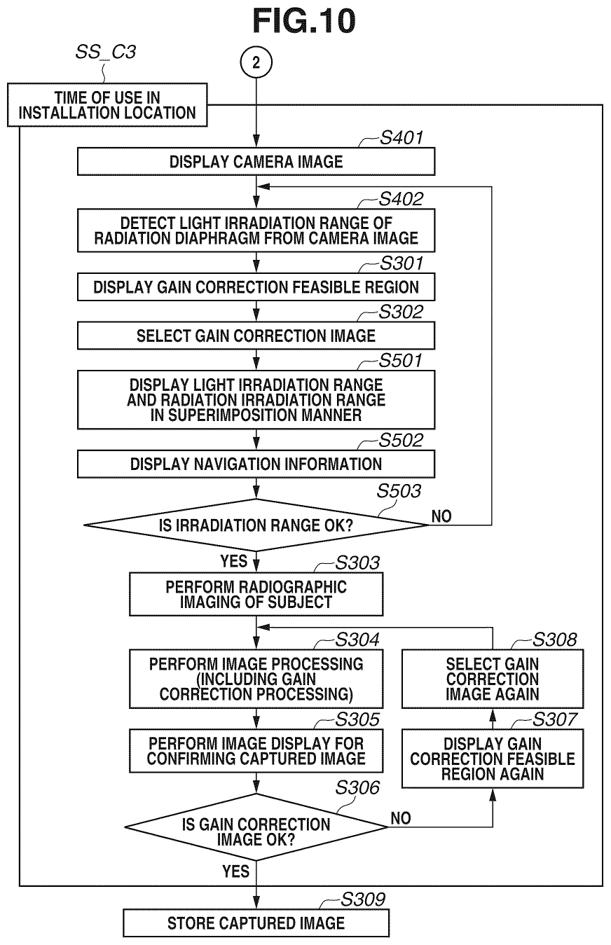

FIG. 10 is a flowchart illustrating an example of a processing procedure performed at the time of use in installation location in a control method for the radiation imaging system 1000 according to the third exemplary embodiment. In FIG. 10, a flowchart illustrating an example of a processing procedure performed at the time of use in installation location in the third exemplary embodiment is denoted by SS_C3. The processing in the flowchart (SS_C3) at the time of use in installation location illustrated in FIG. 10 can be performed after the processing in the flowchart (SS_B1) at the time of installation or at the time of periodic inspection illustrated in FIG. 2B ends. In the processing in the flowchart illustrated in FIG. 10, processing steps similar to those in the processing in the flowchart illustrated in FIG. 8 are assigned the respective same step numbers as those in FIG. 8, and the detailed description thereof is omitted here.

First, as with step S401 illustrated in FIG. 8, the radiation imaging system 1000 displays, in the screen 701 on the display unit 1170, a camera image captured by the visible light camera 1040 mounted to the radiation diaphragm 1020.

Next, as with step S402 illustrated in FIG. 8, the radiation imaging system 1000 (for example, the CPU 1140) detects a light irradiation range 712 of light emitted from the light source of the radiation diaphragm 1020, from the camera image acquired in step S401.

Next, as with step S301 illustrated in FIG. 8 (FIG. 2C), the radiation imaging system 1000 displays a gain correction feasible region on the display unit 1170. It is desirable that a gain correction feasible region in the large-sized radiation imaging unit 1030 be displayed in a screen on the display unit 1170.

Next, as with step S302 illustrated in FIG. 8 (FIG. 2C), the gain correction data selection unit 1120 selects a gain correction image to be used from among a plurality of gain correction images based on, for example, an operation input to the operation panel 1160.

Next, in step S501, for example, the CPU 1140 displays, in the camera image displayed in the screen 701, the light irradiation range 712 and the radiation irradiation range 912, which corresponds to the radiation irradiation range of the gain correction image selected in step S302, in a superimposition manner. To facilitate determining an influence on the actual radiographic image, the CPU 1140 also displays the light irradiation range and the radiation irradiation range in a superimposition manner even in the first correction image 520 and the second correction image 530 in the screen 702 illustrated in FIGS. 9A and 9B.

Next, in step S502, for example, the CPU 1140 displays navigation information. In the example illustrated in FIG. 9A (also in FIG. 9B), the CPU 1140 displays navigation information 913, navigation information 922, and navigation information 932.

Next, in step S503, for example, the CPU 1140 determines whether the light irradiation range acquired in step S402 is contained in a radiation irradiation range for the gain correction image selected in step S302 (OK).

If, in step S503, it is determined that the light irradiation range acquired in step S402 is not contained in a radiation irradiation range for the gain correction image selected in step S302 (NO in step S503), the radiation imaging system 1000 returns the processing to step S402. Then, in step S402, after the radiation diaphragm 1020 is adjusted, the radiation imaging system 1000 performs processing operations in step S402 and subsequent steps again.

On the other hand, if, in step S503, it is determined that the light irradiation range acquired in step S402 is contained in a radiation irradiation range for the gain correction image selected in step S302 (YES in step S503), the radiation imaging system 1000 advances the processing to step S303. Processing operations in step S303 and subsequent steps are similar to the corresponding processing operations illustrated in FIG. 8 (FIG. 2C), and, therefore, the detailed description thereof is omitted here.

In the third exemplary embodiment, the display unit 1170 displays, in a comparable manner, a light irradiation range of light emitted from the light source of the radiation diaphragm 1020 to the radiation imaging unit 1030 and a plurality of radiation irradiation ranges for correction in the radiation imaging unit 1030 (FIGS. 9A and 9B). The display unit 1170 further displays information indicating whether the light irradiation range is contained in the radiation irradiation range for correction (“NG” or “OK” of navigation information 913, navigation information 922, and navigation information 932 illustrated in FIGS. 9A and 9B). Additionally, in a case where the light irradiation range is not contained in the radiation irradiation range for correction, the display unit 1170 is configured to perform the following display. Specifically, the display unit 1170 further displays information indicating a deviation amount relative to the radiation irradiation range for correction in an excess range, which is a portion of the light irradiation range not contained in the radiation irradiation range for correction (arrows in navigation information 913, navigation information 922, and navigation information 932 illustrated in FIGS. 9A and 9B).

According to such a configuration, in addition to obtaining the advantageous effect in the first exemplary embodiment, it is possible to readily recognize a difference between the light irradiation range and the radiation irradiation range for a correction image even on a different axis.

Next, a fourth exemplary embodiment is described. In the description of the fourth exemplary embodiment described below, particulars in common with those in the above-described first to third exemplary embodiments are omitted from description, and only particulars different from those in the above-described first to third exemplary embodiments are described.

The outline configuration of a radiation imaging system according to the fourth exemplary embodiment is similar to the outline configuration of the radiation imaging system 1000 according to the first exemplary embodiment illustrated in FIG. 1. In the fourth exemplary embodiment, a configuration in a case where, after a radiographic image of the subject H is captured, a radiation irradiation range for a correction image is not appropriate is described.

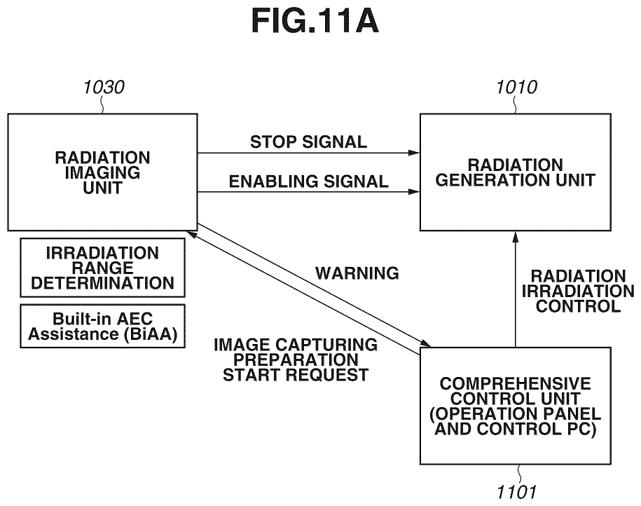

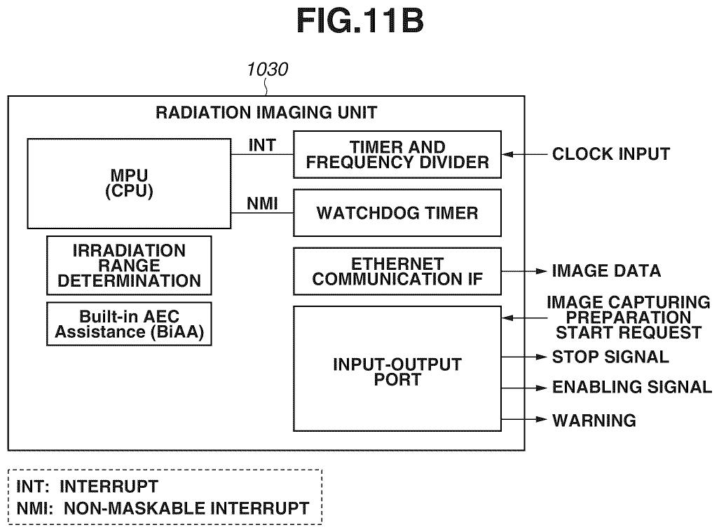

FIG. 11A is a diagram illustrating a first configuration example of a control system for the radiation imaging system 1000 according to the fourth exemplary embodiment. In FIG. 11A, constituent elements similar to the constituent elements illustrated in FIG. 1 are assigned the respective same reference characters as those in in FIG. 1, and the detailed description thereof is omitted here.

The control system illustrated in FIG. 11A includes a radiation generation unit 1010, a radiation imaging unit 1030, and a comprehensive control unit 1101, which is configured with the operation panel 1160 illustrated in FIG. 1 and a control personal computer (PC) including the CPU 1140 and the main memory 1150.

In the control system illustrated in FIG. 11A, when an image capturing preparation start request is output from the comprehensive control unit 1101 including the operation panel 1160 to the radiation imaging unit 1030, the radiation imaging unit 1030 performs preparatory driving. Then, upon the completion of preparation, the radiation imaging unit 1030 outputs an enabling signal for irradiation of radiation R to the radiation generation unit 1010. The radiation generation unit 1010 starts irradiation of radiation R based on the reception of the enabling signal and radiation irradiation control received from the comprehensive control unit 1101 caused by the pressing operation on an irradiation switch performed by the user. The radiation imaging unit 1030 acquires a radiation irradiation range by, for example, starting non-destructive readout of a radiographic image signal. In a case where the radiation irradiation range for a correction image is narrower than the radiation irradiation range for a radiographic image which is being captured, the radiation imaging unit 1030 outputs a stop signal for irradiation of radiation R to the radiation generation unit 1010. Upon receiving the stop signal, the radiation generation unit 1010 immediately stops irradiation of radiation R.

FIG. 11B is a diagram illustrating a second configuration example of the control system for the radiation imaging system 1000 according to the fourth exemplary embodiment. Specifically, FIG. 11B is a diagram illustrating a configuration example of a control system for the radiation imaging unit 1030 according to the fourth exemplary embodiment. In FIG. 11B, constituent elements similar to the constituent elements illustrated in FIG. 1 and FIG. 11A are assigned the respective same reference characters as those in in FIG. 1 and FIG. 11A, and the detailed description thereof is omitted here.

The control system for the radiation imaging unit 1030 illustrated in FIG. 11B includes a micro processing unit (MPU) (CPU), a timer and frequency divider, a watchdog timer, an Ethernet communication interface (IF), and an input-output port. In the control system for the radiation imaging unit 1030 illustrated in FIG. 11B, with regard to the stop signal, to make a reaction as fast as possible, it is desirable that a clock signal which is input to the timer and frequency divider have a high frequency. Since, in a case where there is no reaction for a specific time, some error may be occurring in real-time processing, the watchdog timer is mounted. To the watchdog timer, non-maskable interrupt (NMI) of the MPU (CPU) is made. With this interrupt, even if an image capturing preparation start request from the operation panel 1160 is input to the radiation imaging unit 1030, a warning signal indicating that an error is occurring in real-time processing is output to the operation panel 1160. Image data acquired by the radiation imaging unit 1030 is output via, for example, the gigabit Ethernet communication IF. The signal for image capturing preparation start request, the stop signal, the enabling signal, and the warning signal are input and output via the input-output port.

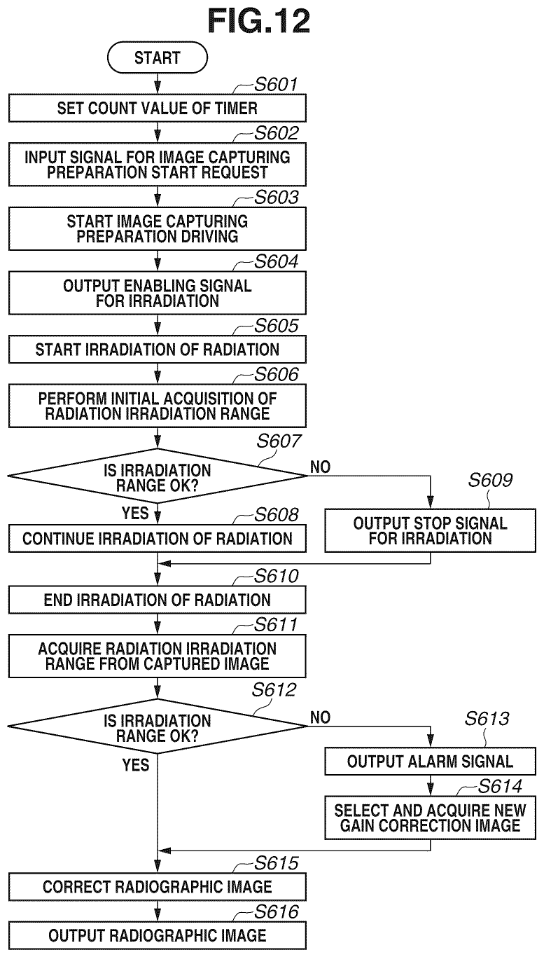

FIG. 12 is a flowchart illustrating an example of a processing procedure in a control method for the radiation imaging system 1000 according to the fourth exemplary embodiment. Specifically, FIG. 12 is a flowchart illustrating an example of a processing procedure in a control method for the control system of the radiation imaging system 1000 according to the fourth exemplary embodiment illustrated in FIG. 11A (also FIG. 11B).

First, in step S601, the radiation imaging unit 1030 sets a count value of the timer. The count value of the timer to which an initial value has been set is made to be decremented by the timer tempered by a frequency divider according to a clock input.

Next, in step S602, the radiation imaging unit 1030 receives inputting of a signal for image capturing preparation start request. Here, the signal for image capturing preparation start request is input from the comprehensive control unit 1101 including the operation panel 1160 to the radiation imaging unit 1030.

Next, in step S603, the radiation imaging unit 1030 starts image capturing preparation driving. For example, the radiation imaging unit 1030 starts preparation driving such as void reading of a signal.

Next, in step S604, the radiation imaging unit 1030 outputs an enabling signal for irradiation of radiation R to the radiation generation unit 1010. After performing the image capturing preparation driving for a predetermined time or a predetermined number of times and becoming able to capture a stable radiographic image, the radiation imaging unit 1030 outputs an enabling signal for irradiation of radiation R to the radiation generation unit 1010.

Next, in step S605, the radiation generation unit 1010 starts irradiation of radiation R based on the enabling signal received from the radiation imaging unit 1030 and radiation irradiation control received from the comprehensive control unit 1101 caused by the pressing operation on an irradiation switch performed by the user.

Next, in step S606, the radiation imaging unit 1030 performs initial acquisition of a radiation irradiation range by non-destructive reading. While, here, the acquisition of a radiation irradiation range to be performed during irradiation of radiation by non-destructive reading has been described, a configuration in which the radiation imaging unit 1030 acquires a radiation irradiation range by reading out, for example, a bias signal for only predetermined lines can be employed.

Next, in step S607, the radiation imaging unit 1030 determines whether the radiation irradiation range acquired in step S606 is contained in the radiation irradiation range for a correction image (OK).

If, in step S607, it is determined that the radiation irradiation range acquired in step S606 is contained in the radiation irradiation range for a correction image (YES in step S607), the radiation imaging unit 1030 advances the processing to step S608. In step S608, the radiation imaging unit 1030 causes the radiation generation unit 1010 to continue irradiation of radiation R.

On the other hand, if, in step S607, it is determined that the radiation irradiation range acquired in step S606 is not contained in the radiation irradiation range for a correction image (NO in step S607), the radiation imaging unit 1030 advances the processing to step S609. In step S609, the radiation imaging unit 1030 outputs a stop signal for irradiation of radiation R to the radiation generation unit 1010. For example, in a case where the radiation irradiation range acquired in step S606 is larger than the radiation irradiation range for a correction image and, particularly, in a case where a region of interest is larger than the radiation irradiation range for a correction image, the radiation imaging unit 1030 outputs a stop signal for irradiation of radiation R to the radiation generation unit 1010. After that, for example, the radiation generation control unit 1070 performs control to stop irradiation of radiation R from the radiation generation unit 1010, based on the stop signal for irradiation of radiation R output from the radiation imaging unit 1030.

In a case where the processing operation in step S608 has ended or in a case where the processing operation in step S609 has ended, the radiation imaging unit 1030 advances the processing to step S610. In step S610, the radiation generation unit 1010 ends irradiation of radiation R. After the end of irradiation of radiation R, a radiographic image obtained by the radiation imaging unit 1030 is output to the comprehensive control unit 1101 including the control PC via, for example, the Ethernet communication IF.

Next, in step S611, for example, the comprehensive control unit 1101 acquires a radiation irradiation range from the received radiographic image. Here, the comprehensive control unit 1101 acquires a radiation irradiation range by, for example, image analysis with the radiographic image used as an input.

Next, in step S612, for example, the comprehensive control unit 1101 determines whether the radiation irradiation range for the radiographic image acquired in step S611 is broader than the radiation irradiation range for a correction image (OK). Since, as described above with reference to FIGS. 5A and 5B, there may be somewhat an error between the light irradiation range and the radiation irradiation range, the comprehensive control unit 1101 performs such determination based on the finally acquired radiographic image.

If, in step S612, it is determined that the radiation irradiation range for the radiographic image acquired in step S611 is not broader than the radiation irradiation range for a correction image (NO in step S612), the comprehensive control unit 1101 advances the processing to step S613. In step S613, for example, the comprehensive control unit 1101 outputs an alarm signal concerning warning. While, in response to the alarm signal being output, the user recognizes that the correction image is not appropriate, performing image capturing of the subject H again leads to wasteful radiation R being radiated.

Next, in step S614, for example, the comprehensive control unit 1101 selects and acquires a new correction image (gain correction image) based on, for example, an operation input to the operation panel 1160. To avoid irradiation of wasteful radiation R, after performing image capturing of the subject H, the comprehensive control unit 1101 selects and acquires a new correction image (gain correction image). For example, the comprehensive control unit 1101 selects and acquires a new correction image (gain correction image) for the time of factory shipment. Outputting an alarm signal immediately after image capturing of the subject H enables determining the appropriateness of a correction image. Therefore, directly acquiring, immediately after image capturing of the subject H, a correction image without moving the radiation generation unit 1010 and the radiation diaphragm 1020 or the radiation imaging unit 1030 enables acquiring the newest correction image.

In a case where the processing operation in step S614 has ended or if, in step S612, it is determined that the radiation irradiation range for the radiographic image acquired in step S611 is broader than the radiation irradiation range for a correction image (YES in step S612), the comprehensive control unit 1101 advances the processing to step S615. In step S615, for example, the comprehensive control unit 1101 corrects (gain-corrects) the radiographic image obtained by image capturing, with use of the currently selected correction image (gain correction image).

Next, in step S616, for example, the comprehensive control unit 1101 outputs the radiographic image subjected to image correction processing.

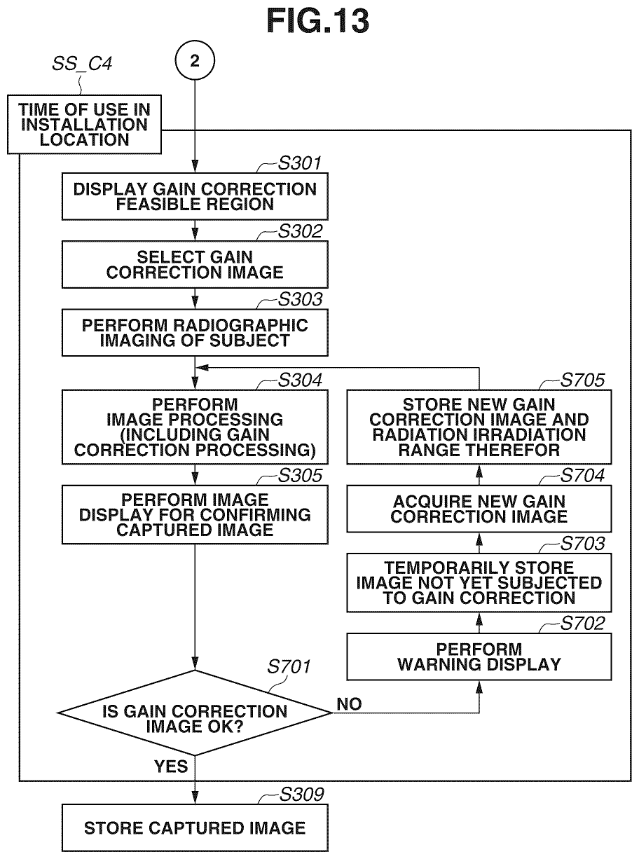

FIG. 13 is a flowchart illustrating an example of a processing procedure performed at the time of use in installation location in the control method for the radiation imaging system 1000 according to the fourth exemplary embodiment. In FIG. 13, a flowchart illustrating an example of a processing procedure performed at the time of use in installation location in the fourth exemplary embodiment is denoted by SS_C4. The processing in the flowchart (SS_C4) at the time of use in installation location illustrated in FIG. 13 can be performed after the processing in the flowchart (SS_B1) at the time of installation or at the time of periodic inspection illustrated in FIG. 2B ends. In the processing in the flowchart illustrated in FIG. 13, processing steps similar to those in the processing in the flowchart illustrated in FIG. 2C are assigned the respective same step numbers as those in FIG. 2C, and the detailed description thereof is omitted here.

First, as with step S301 illustrated in FIG. 2C, the radiation imaging system 1000 displays a gain correction feasible region on the display unit 1170. It is desirable that a gain correction feasible region in the large-sized radiation imaging unit 1030 be displayed in a screen on the display unit 1170.

Next, as with step S302 illustrated in FIG. 2C, the gain correction data selection unit 1120 selects a gain correction image to be used from among a plurality of gain correction images based on, for example, an operation input to the operation panel 1160.

Next, as with step S303 illustrated in FIG. 2C, the radiation imaging system 1000 performs radiographic imaging of the subject H.

Next, as with step S304 illustrated in FIG. 2C, the image correction unit 1090 performs image processing such as performing gain correction of the radiographic image captured in step S303 with use of the gain correction image selected in step S302.

Next, as with step S305 illustrated in FIG. 2C, the display unit 1170 performs image display for confirming the radiographic image (captured image) subjected to image processing in step S304.

Next, in step S701, for example, the CPU 1140 or the gain correction determination unit 1130 determines whether the currently selected gain correction image is OK based on, for example, an operation input to the operation panel 1160. Here, in the processing operation in step S306 illustrated in FIG. 2C, as the gain correction image, a previously acquired and stored one is used (including the one stored in step S108 illustrated in FIG. 2A). On the other hand, in the processing operation in step S701 illustrated in FIG. 13, a new gain correction image acquired in step S704 described below is also included.

If, in step S701, it is determined that the currently selected gain correction image is not OK (i.e., is NG) (NO in step S701), the CPU 1140 or the gain correction determination unit 1130 advances the processing to step S702. In step S702, the radiation imaging system 1000 determines that there is no selectable appropriate gain correction image and then performs warning display of that effect on the display unit 1170.

Next, in step S703, the radiation imaging system 1000 temporarily stores an image (radiographic image) not yet subjected to gain correction in, for example, the storage unit 1100.

Next, in step S704, the radiation imaging system 1000 acquires a new gain correction image. It is desirable that the new gain correction image be acquired with use of the same geometric layout and radiation irradiation condition (such as focus size, kilovolts peak (kVp), and milliampere-second (mAs)) as those for image capturing of the subject H in step S303.

Next, in step S705, the radiation imaging system 1000 stores the new gain correction image acquired in step S704 in, for example, the second gain correction data storing unit 1052. After that, the radiation imaging system 1000 returns the processing to step S304, thus performing processing operations in step S304 and subsequent steps with use of the new gain correction image acquired in step S704.

If, in step S701, it is determined that the currently selected gain correction image is OK (YES in step S701), the CPU 1140 or the gain correction determination unit 1130 advances the processing to step S309. In step S309, the radiation imaging system 1000 stores the captured radiographic image.

In the fourth exemplary embodiment, as compared with the above-described first exemplary embodiment, for example, in a case where image capturing of the subject His performed with the outermost area of the effective pixel range of the large-sized radiation imaging unit 1030, the image quality may increase. In the above-described first exemplary embodiment, with respect to a gain correction image for the time of factory shipment to be stored in step S108 illustrated in FIG. 2A, the center of the radiation generation unit 1010 is often fitted to the center of the large-sized radiation imaging unit 1030. On the other hand, in the fourth exemplary embodiment, in step S704 illustrated in FIG. 13, the new gain correction image can be acquired with use of the same geometric layout and radiation irradiation condition (such as focus size, kVp, and mAs) as those for image capturing of the subject H. This is because not only geometric conditions but also radiation irradiation conditions are close to each other between the actual image capturing of the subject H and the gain correction image to be used. Therefore, in a case where image capturing is performed at a position close to the inner end of the effective pixel range of the large-sized radiation imaging unit 1030, the fourth exemplary embodiment can be more appropriate.

In the radiation imaging system 1000 according to the above-described fourth exemplary embodiment, in a case where there is no selectable appropriate gain correction image, the display unit 1170 performs warning display (step S702 illustrated in FIG. 13). In the radiation imaging system 1000 according to the fourth exemplary embodiment, in a case where the radiation irradiation range for image capturing is not appropriate, the radiation generation control unit 1070 performs control to stop irradiation of radiation R (step S609 illustrated in FIG. 12).

In the radiation imaging system 1000 according to the fourth exemplary embodiment, as with the first exemplary embodiment, even in a case where it is not possible to radiate radiation R to the whole area of the radiation imaging unit 1030, it is possible to appropriately correct the captured radiographic image.

Next, a fifth exemplary embodiment is described. In the description of the fifth exemplary embodiment described below, particulars in common with those in the above-described first to fourth exemplary embodiments are omitted from description, and only particulars different from those in the above-described first to fourth exemplary embodiments are described.

The outline configuration of a radiation imaging system according to the fifth exemplary embodiment is similar to the outline configuration of the radiation imaging system 1000 according to the first exemplary embodiment illustrated in FIG. 1. In the fifth exemplary embodiment, a configuration in which an example of application of the radiation imaging system 1000 according to the fifth exemplary embodiment is illustrated is described.

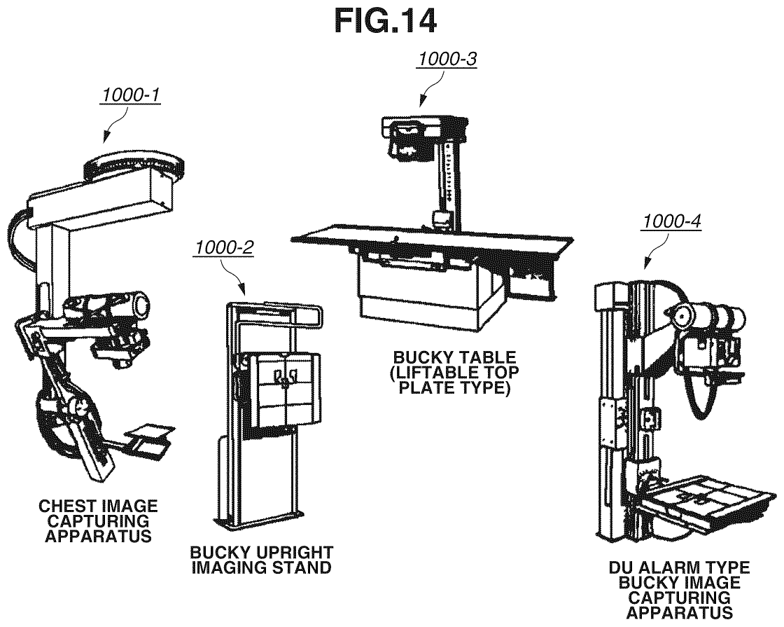

FIG. 14 is a diagram illustrating examples of application of the radiation imaging system 1000 according to the fifth exemplary embodiment. A radiation imaging system 1000-1 according to a first example of application of the fifth exemplary embodiment is obtained by applying the radiation imaging system 1000 according to any one of the first to fourth exemplary embodiments to a chest image capturing apparatus. A radiation imaging system 1000-2 according to a second example of application of the fifth exemplary embodiment is obtained by applying the radiation imaging system 1000 according to any one of the first to fourth exemplary embodiments to a bucky upright imaging stand. A radiation imaging system 1000-3 according to a third example of application of the fifth exemplary embodiment is obtained by applying the radiation imaging system 1000 according to any one of the first to fourth exemplary embodiments to a bucky table (liftable top plate type). A radiation imaging system 1000-4 according to a fourth example of application of the fifth exemplary embodiment is obtained by applying the radiation imaging system 1000 according to any one of the first to fourth exemplary embodiments to a DU alarm type bucky image capturing apparatus.

While, in the above-described exemplary embodiments, an example in which a gain correction image is applied as an example of a correction image in the present disclosure has been described, the present disclosure is not limited to the gain correction image, but can also be applied to a correction image concerning other image processing.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random access memory (RAM), a read-only memory (ROM), a storage of distributed computing systems, an optical disk (such as a compact disc (CD), digital versatile disc (DVD), or Blu-ray Disc (BD)™), a flash memory device, a memory card, and the like.

Each of the above-described exemplary embodiments merely represents a specific example in implementing the present disclosure, and should not be construed to limit the technical scope of the present disclosure. Thus, the present disclosure can be implemented in various forms without departing from the technical idea thereof or the principal features thereof.

The disclosure in each of the above-described exemplary embodiments includes, for example, the following configurations, method, and storage medium.

### <Configuration 1>

A radiation imaging system including:
a radiation imaging unit configured to capture a radiographic image based on incident radiation;
an acquisition unit configured to acquire a radiation irradiation range for image capturing, which is an irradiation range of the radiation used when the radiographic image has been captured by the radiation imaging unit; and
a display unit configured to display, in a selectable manner, at least one correction image from a plurality of correction images different in irradiation condition of the radiation, based on the radiation irradiation range for image capturing.

### <Configuration 2>

The radiation imaging system as set forth in Configuration 1, further including a storing unit configured to store a radiation irradiation range for correction, which is an irradiation range of the radiation used when each correction image from the plurality of correction images has been acquired by the radiation imaging unit,
wherein the display unit displays, in a selectable manner, the at least one correction image from the plurality of correction images based on the radiation irradiation range for image capturing and a plurality of radiation irradiation ranges for correction each corresponding to the radiation irradiation range for correction in the plurality of correction images.

### <Configuration 3>

The radiation imaging system as set forth in Configuration 2,
wherein the radiation irradiation range for image capturing is acquired by performing image analysis of the radiographic image, and
wherein the radiation irradiation range for correction is acquired by performing image analysis of the correction image.

### <Configuration 4>

The radiation imaging system as set forth in Configuration 2 or 3, wherein at least one radiation irradiation range from the radiation irradiation range for image capturing and the radiation irradiation range for correction is acquired with use of one or more pieces of information from information about a focus position of a radiation generation unit which generates the radiation, information about a relative position between the radiation generation unit and the radiation imaging unit, information about a relative angle between the radiation generation unit and the radiation imaging unit, information about an image capturing distance between the radiation generation unit and the radiation imaging unit, or information about an aperture opening degree of a radiation diaphragm provided between the radiation generation unit and the radiation imaging unit.

### <Configuration 5>

The radiation imaging system as set forth in any one of Configuration 2 to 4, wherein the display unit displays, in a comparable manner, the radiation irradiation range for image capturing in the radiation imaging unit and the plurality of radiation irradiation ranges for correction in the radiation imaging unit.

### <Configuration 6>

The radiation imaging system as set forth in Configuration 5, wherein the display unit further displays information about at least one of an anode or a cathode of a radiation generation unit which generates the radiation.

### <Configuration 7>

The radiation imaging system as set forth in Configuration 5 or 6, further including a radiation diaphragm provided with a light source between a radiation generation unit which generates the radiation and the radiation imaging unit and configured to adjust an irradiation field of the radiation,
wherein the display unit further displays, in a comparable manner, a light irradiation range, which is an irradiation range of light emitted from the light source of the radiation diaphragm to the radiation imaging unit, and the plurality of radiation irradiation ranges for correction in the radiation imaging unit.

### <Configuration 8>

The radiation imaging system as set forth in Configuration 7, further including a camera mounted to the radiation diaphragm,
wherein the light irradiation range is displayed as a camera image obtained by the camera capturing an image of the radiation imaging unit irradiated with the light.

### <Configuration 9>

The radiation imaging system as set forth in Configuration 8,
wherein the plurality of radiation irradiation ranges for correction is displayed as the plurality of correction images, and
wherein an index indicating a placement position of the radiation imaging unit is depicted in each of the camera image and the plurality of correction images.

### <Configuration 10>

The radiation imaging system as set forth in any one of Configuration 7 to 9, wherein the display unit further displays information indicating whether the light irradiation range is contained in the radiation irradiation range for correction.

### <Configuration 11>

The radiation imaging system as set forth in any one of Configuration 7 to 10, wherein the display unit further displays, in a case where the light irradiation range is not contained in the radiation irradiation range for correction, information indicating that effect and, additionally, information indicating a deviation amount relative to the radiation irradiation range for correction in an excess range, which is a portion of the light irradiation range not contained in the radiation irradiation range for correction.

### <Configuration 12>

The radiation imaging system as set forth in any one of Configuration 7 to 11, wherein the display unit further displays a screen for use in calibrating the light irradiation range.

### <Configuration 13>

The radiation imaging system as set forth in any one of Configuration 7 to 12, wherein the display unit performs warning display in a case where there is no selectable appropriate correction image from the plurality of correction images.

### <Configuration 14>

The radiation imaging system as set forth in any one of Configuration 1 to 13, further including a control unit configured to stop irradiation of the radiation in a case where the radiation irradiation range for image capturing is not appropriate.

### <Configuration 15>

The radiation imaging system as set forth in any one of Configuration 1 to 14, further including a correction unit configured to correct the radiographic image with use of a correction image selected from among the at least one correction image displayed in a selectable manner by the display unit.

### <Configuration 16>

The radiation imaging system as set forth in any one of Configuration 1 to 15, wherein the correction image is a gain correction image for use in correcting a difference in gain of the radiation imaging unit.

### <Configuration 17>

The radiation imaging system as set forth in any one of Configuration 1 to 16, wherein the radiation imaging unit has a rectangular effective pixel range with a long side length of 18 inches or more and a diagonal length of 25 inches or more.

### <Method 1>

A control method for a radiation imaging system including a radiation imaging unit configured to capture a radiographic image based on incident radiation, the control method including:
acquiring a radiation irradiation range for image capturing, which is an irradiation range of the radiation used when the radiographic image has been captured by the radiation imaging unit; and
displaying, in a selectable manner, at least one correction image from a plurality of correction images different in irradiation condition of the radiation, based on the radiation irradiation range for image capturing.

### <Storage Medium 1>

A non-transitory computer-readable storage medium storing computer-executable instructions that, when executed by a computer, cause the computer to perform a method for a radiation imaging system including a radiation imaging unit configured to capture a radiographic image based on incident information, the method including:
acquiring a radiation irradiation range for image capturing, which is an irradiation range of the radiation used when the radiographic image has been captured by the radiation imaging unit; and
displaying, in a selectable manner, at least one correction image from a plurality of correction images different in irradiation condition of the radiation, based on the radiation irradiation range for image capturing.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2024-002797 filed Jan. 11, 2024, which is hereby incorporated by reference herein in its entirety.

What is claimed is:

1. A radiation imaging system comprising:
one or more controllers configured to:
acquire information about a radiation irradiation range for radiographic imaging; and
cause at least one correction image from a plurality of correction images different in irradiation condition of radiation to be displayed on a display in a selectable manner based on the information about the radiation irradiation range for radiographic imaging.

2. The radiation imaging system according to claim 1, wherein the one or more controllers are further configured to:
store information about a radiation irradiation range for correction, which is an irradiation range of the radiation used when each of the plurality of correction images has been acquired; and
display, in a selectable manner, the at least one correction image based on the information about the radiation irradiation range for radiographic imaging and the information about the radiation irradiation range for correction.

3. The radiation imaging system according to claim 2,
wherein the information about the radiation irradiation range for radiographic imaging is acquired by performing image analysis of a radiographic image captured based on incident radiation, and
wherein the information about the radiation irradiation range for correction is acquired by performing image analysis of the correction image.

4. The radiation imaging system according to claim 2, wherein information about at least one radiation irradiation range from the radiation irradiation range for radiographic imaging and the radiation irradiation range for correction is acquired using one or more pieces of information from information about a focus position of a radiation generation unit that generates the radiation, information about a relative position between the radiation generation unit and a radiation imaging unit which captures a radiographic image based on incident radiation, information about a relative angle between the radiation generation unit and the radiation imaging unit, information about an image capturing distance between the radiation generation unit and the radiation imaging unit, or information about an aperture opening degree of a radiation diaphragm provided between the radiation generation unit and the radiation imaging unit.

5. The radiation imaging system according to claim 2, wherein, on the display, the radiation irradiation range for radiographic imaging in a radiation imaging unit that captures a radiographic image based on incident radiation and a plurality of radiation irradiation ranges for correction each corresponding to the radiation irradiation range for correction in the radiation imaging unit are displayed in a comparable manner.

6. The radiation imaging system according to claim 5, wherein, on the display, information about at least one of an anode or a cathode of a radiation generation unit that generates the radiation is displayed.

7. The radiation imaging system according to claim 5, further comprising:
a radiation generation unit configured to generate radiation;
the radiation imaging unit; and
a radiation diaphragm provided with a light source between the radiation generation unit and the radiation imaging unit and configured to adjust an irradiation field of the radiation,
wherein, on the display, a light irradiation range, which is an irradiation range of light emitted from the light source of the radiation diaphragm to the radiation imaging unit, and the plurality of radiation irradiation ranges for correction in the radiation imaging unit are displayed in a comparable manner.

8. The radiation imaging system according to claim 7, further comprising a camera mounted to the radiation diaphragm, wherein the light irradiation range is displayed as a camera image obtained by the camera capturing an image of the radiation imaging unit irradiated with the light.

9. The radiation imaging system according to claim 8,
wherein the plurality of radiation irradiation ranges for correction is displayed as the plurality of correction images, and
wherein an index indicating a placement position of the radiation imaging unit is depicted in each of the camera image and the plurality of correction images.

10. The radiation imaging system according to claim 7, wherein, on the display, information indicating whether the light irradiation range is contained in the radiation irradiation range for correction is displayed.

11. The radiation imaging system according to claim 7, wherein, on the display, in a case where the light irradiation range is not contained in the radiation irradiation range for correction, information indicating that light irradiation is not contained and information indicating a deviation amount relative to the radiation irradiation range for correction in an excess range, which is a portion of the light irradiation range not contained in the radiation irradiation range for correction are displayed.

12. The radiation imaging system according to claim 7, wherein, on the display, a screen for use in calibrating the light irradiation range is displayed.

13. The radiation imaging system according to claim 1, wherein, on the display, a warning display is provided in a case where there is no selectable appropriate correction image from the plurality of correction images.

14. The radiation imaging system according to claim 1, wherein the one or more controllers are further configured to perform control to stop irradiation of the radiation in a case where the radiation irradiation range for radiographic imaging is not appropriate.

15. The radiation imaging system according to claim 1, wherein the one or more controllers are further configured to correct a radiographic image captured based on incident radiation using a correction image selected from among the at least one correction image displayed in a selectable manner on the display.

16. The radiation imaging system according to claim 1, wherein the correction image is a gain correction image used in correcting a difference in gain of a radiation imaging unit that captures a radiographic image based on incident radiation.

17. The radiation imaging system according to claim 1, wherein a radiation imaging unit that captures a radiographic image based on incident radiation has a rectangular effective pixel range with a long side length of 18 inches or more and a diagonal length of 25 inches or more.

18. A method for controlling a radiation imaging system including a radiation imaging unit that captures a radiographic image based on incident radiation, the method comprising:
acquiring information about a radiation irradiation range for radiographic imaging; and
causing at least one correction image from a plurality of correction images different in irradiation condition of radiation to be displayed on a display in a selectable manner based on the information about the radiation irradiation range for radiographic imaging.

19. A non-transitory computer-readable storage medium storing computer-executable instructions that, when executed by a computer, cause the computer to perform a method for a radiation imaging system including a radiation imaging unit configured to capture a radiographic image based on incident information, the method comprising:
acquiring information about a radiation irradiation range for radiographic imaging; and
causing at least one correction image from a plurality of correction images different in irradiation condition of radiation to be displayed on a display in a selectable manner based on the information about the radiation irradiation range for radiographic imaging.
